(19) United States
(12) Patent Application Publication
(10) Pub. No.: US 2026/0007861 A1
(43) Pub. Date: Jan. 8, 2026

(54) STABILIZER CLAMP APPARATUS FOR USE WITH IMPLANT DELIVERY APPARATUS

(71) Applicant: EDWARDS LIFESCIENCES CORPORATION, Irvine, CA (US)

(72) Inventor: Kurt Kelly Reed, Costa Mesa, CA (US)

(21) Appl. No.: 19/325,343

(22) Filed: Sep. 10, 2025

Related U.S. Application Data
(63) Continuation of application No. PCT/US2024/019784, filed on Mar. 13, 2024.
(60) Provisional application No. 63/490,143, filed on Mar. 14, 2023.

(51) Int. Cl. A61M 25/02; A61B 90/57

(52) U.S. Cl. CPC A61M 25/02; A61B 90/57; A61M 2025/024

(57) ABSTRACT

Devices, systems, and methods for stabilizing delivery apparatus and/or guide catheter apparatus during delivery and implantation of a prosthetic implant are disclosed. Each of the delivery and guide catheter apparatus can include a handle and a shaft (delivery shaft or guide catheter shaft) extending distally from the handle. A distal cap portion of each of the handles can be coupled to a stabilizer clamp of a stabilizer system to retain a position of the handles relative to each other in an axial direction. The stabilizer clamps are configured to enable rotation of the distal cap therein so that torque applied to a handle is transmitted to a respective shaft. A jaw portion of the stabilizer clamp can be configured to balance or equalize toque resistance for rotation of the handle in a first (for example, clockwise) direction relative to a second opposing (for example, counterclockwise) direction.

### CROSS-REFERENCE TO RELATED APPLICATIONS

This application is a continuation of PCT patent application no. CT/US2024/019784, filed on Mar. 13, 2024, which application claims the benefit of U.S. Provisional Patent Application No. 63/490,143, filed Mar. 14, 2023, each of these applications being incorporated herein in its entirety by this specific reference.

### FIELD

The present disclosure relates to delivery apparatus for prosthetic medical devices and stabilizer apparatus for use with the delivery apparatus.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery or femoral vein) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A guide catheter (which can also be referred to as a guide sheath) can be used for introducing a delivery apparatus, such as the prosthetic heart valve delivery apparatus described above, into the patient's vasculature. The guide catheter can include an elongated shaft that is inserted into the vasculature and a handle that remains outside the patient and can be used to manipulate the shaft. The delivery apparatus can be pushed through a main lumen of the guide catheter in order to help navigate the delivery apparatus to a target implantation site within the patient. The delivery apparatus can also include a handle that remains outside the patient and can be used to manipulate the delivery apparatus. The guide catheter and the delivery apparatus can be controlled via rotation of their respective handles and/or actuation of one or more actuators or knobs on the respective handles.

### SUMMARY

Described herein are prosthetic heart valves, docking devices, delivery apparatus, guide catheter apparatus, implant delivery systems, and methods for utilizing such apparatus and systems for implanting prosthetic heart valves. A delivery apparatus and a guide catheter apparatus can each comprise a handle and one or more shafts coupled to the respective handle. The disclosed systems and methods can, for example, provide stable positioning and control of one or more delivery apparatus or guide catheter apparatus handles during an implantation procedure, such that the positioning of the prosthetic implant(s) can be adjusted and/or maintained during the implantation procedure via control of respective handles. As such, the devices and methods disclosed herein can, among other things, overcome one or more of the deficiencies of various known delivery apparatus and systems for implantation of implantable devices.

A delivery apparatus system can include a delivery apparatus and a stabilizer clamp.

In some examples, the stabilizer clamp is configured to receive a portion of a handle of the delivery apparatus.

In some examples, a stabilizer clamp includes a base portion configured to be coupled to or integral with a stabilizer rail or table.

In some examples, the stabilizer clamp includes a jaw portion extending from the base portion and configured to receive the portion of the handle therein.

In some examples, the jaw portion comprises first and second jaw members that each include an interior curved wall, which together define a mouth portion for receiving the portion of the handle.

In some examples, the first jaw member is a moveable jaw member and the second jaw member is a stationary jaw member, the stabilizer clamp including a spring member between the moveable jaw member and the stationary jaw member to bias the moveable jaw member to a closed position of the mouth.

In some examples, the curved interior wall of the moveable jaw member has a reduced surface area relative to the curved interior wall of the stationary jaw member.

In some examples, the curved interior wall of the moveable jaw member has a reduced or recessed lip at the opening of the mouth relative to a lip of the stationary jaw member.

In some examples, the curved interior wall of the moveable jaw member has a surface gradient including a lower friction surface in a region of an upper lip at the opening of the mouth and a higher friction area at a region of a lower lip on an opposing end of the curved interior wall of the moveable jaw member.

In some examples, the stabilizer clamp includes a locking mechanism or lock for locking or limiting movement of the moveable member.

In some examples, the stabilizer clamp is a dual lever stabilizer clamp.

In some examples, each of the first jaw member and the second jaw member is a lever or a moveable jaw member (that is, a first lever and a second lever).

In some examples, the stabilizer clamp includes a mechanism coupled to or between the first jaw member and the second jaw member such that that application of a rotational force towards the open position of the mouth on one of the first lever or the second lever to results in opening of the other of the first lever or the second lever.

In some examples, a minimum torque applied to or required to overcome the clamping force of the stabilizer clamp is at least 21 N-cm.

In some examples, a minimum torque required for rotation of the handle in one or more of the first direction or the second opposing direction is in a range of 15 N-cm to 30 N-cm.

In some examples, the at least one spring member can have a spring rate that is sufficient to resist axial rotation of a handle when no rotational force (such as application of torque on the handle by an operator) acts upon the handle.

In some examples, the at least one spring member can have a spring rate of in a range of 5 lbs./in. to 50 lbs./in., such as, for example, 30 lbs./in.

In some examples, a ratio of torque resistance for rotation of the handle in a first direction relative to torque resistance for rotation of the handle in a second opposing direction is within a range of 0.8 to 1.2.

In some examples, a ratio of torque resistance for rotation of the handle in a first direction relative to torque resistance for rotation of the handle in a second opposing direction is within a range of 0.9 to 1.1.

In some examples, a ratio of torque resistance for rotation of the handle in a first direction relative to torque resistance for rotation of the handle in a second opposing direction is approximately 1.0.

In some examples, a stabilizer system includes a stabilizer table or rail and two or more stabilizer clamps.

In some examples, the stabilizer system is configured to have the handles of a delivery apparatus and a guide catheter apparatus coupled thereto in tandem via the two or more stabilizer clamps.

In some examples, a stabilizer clamp is configured to resist or limit movement of a handle when no rotational force is applied the handle.

In some examples, a stabilizer clamp is configured to resist or limit movement of a handle when a knob or other actuator on the handle is actuated.

In some examples, a stabilizer clamp is configured such that a ratio of torque resistance for rotation of a handle in a first direction relative to torque resistance for rotation of the handle in a second opposing direction is within a range of 0.8 to 1.2.

In some examples, a stabilizer clamp is configured such that a ratio of torque resistance for rotation of the handle in a first direction relative to torque resistance for rotation of the handle in a second opposing direction is within a range of 0.9 to 1.1.

In some examples, a stabilizer clamp is configured such that a ratio of torque resistance for rotation of the handle in a first direction relative to torque resistance for rotation of the handle in a second opposing direction is approximately 1.0.

In some examples, a delivery apparatus stabilizer system or a stabilizer clamp, or a method of use thereof, comprises one or more of the components recited in Examples 1-33 below.

In one representative example, a delivery apparatus system comprising: a delivery apparatus comprising: a handle; and a shaft extending distally from the handle, the shaft configured for delivery of an implantable device; and a stabilizer clamp comprising a base portion and a jaw portion extending from the base portion; wherein the jaw portion comprises: a first jaw member comprising a first curved interior wall; and a second jaw member comprising a second curved interior wall, wherein the first curved interior wall and the second curved interior wall form a mouth configured to receive a portion of the handle, and wherein at least one of the first jaw member or the second jaw member is a lever rotatable around a fulcrum for opening and closing of the mouth; and at least one spring member configured to bias the lever towards a closed position of the mouth; and wherein, when the portion of the handle is received within the mouth, a ratio of torque resistance for rotation of the handle in a first direction relative to torque resistance for rotation of the handle in a second opposing direction is within a range of 0.8 to 1.2.

In another representative example, a stabilizer clamp configured for use with an implant delivery apparatus, the stabilizer clamp comprising: a base portion; and a jaw portion extending from the base portion, the jaw portion comprising: a first jaw member comprising a first curved interior wall; and a second jaw member comprising a second curved interior wall, wherein the first curved interior wall and the second curved interior wall form a mouth configured to receive a portion of a handle of the implant delivery apparatus, and wherein at least one of the first jaw member or the second jaw member is a lever rotatable around a fulcrum for opening and closing of the mouth; and at least one spring member configured to bias the lever towards a closed position of the mouth; and wherein the stabilizer clamp is configured such that, when the portion of the handle is received within the mouth, a ratio of torque resistance for rotation of the handle in a first direction relative to torque resistance for rotation of the handle in a second opposing direction is within a range of 0.8 to 1.2.

In another representative example, a method of operating a delivery apparatus system, the method comprising: inserting a portion of a first handle of a first delivery apparatus into a mouth of a first stabilizer clamp, the first stabilizer clamp comprising a base portion attached to a stabilizer rail or table and a first jaw member and a second jaw member extending from the base portion, the mouth defined by a first curved interior wall of the first jaw member and a second curved interior wall of a second jaw member, wherein at least one of the first jaw member or the second jaw member is a lever rotatable around a fulcrum for opening and closing of the mouth, and wherein the first stabilizer clamp further comprises at least one spring member configured to bias the lever towards a closed position of the mouth; rotating the first handle in a first direction to transmit torque to a first shaft coupled to the first handle; and rotating the first handle in a second opposing direction to transit torque to the first shaft coupled to the first handle; wherein the first stabilizer clamp is configured such that, when the portion of the first handle is received within the mouth thereof, a ratio of torque resistance for rotation of the first handle in the first direction relative to torque resistance for rotation of the first handle in the second opposing direction is within a range of 0.8 to 1.2.

In some examples, the above method(s) can be performed in connection with an implantation procedure performed on a living animal or on a simulation, such as on a cadaver, cadaver heart, anthropomorphic ghost, simulator (for example, with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 is a side view of a guide catheter configured to receive a delivery apparatus and/or a tool and guide the delivery apparatus and/or the tool through a portion of a patient's vasculature, according to one example.

FIG. 6 is a side view of a delivery apparatus for a docking device, according to an example.

FIG. 7 is a perspective view of a docking device for use with the delivery apparatus of FIG. 6, according to an example.

FIG. 8 is a perspective view of a delivery apparatus for a prosthetic heart valve, according to an example.

FIG. 9 is a perspective view of a prosthetic heart valve for use with the delivery apparatus of FIG. 11, according to an example.

FIGS. 10A and 10B are respectively perspective and top views of an exemplary stabilizer rail system for use with the delivery apparatus disclosed herein, according to an example.

FIG. 11 is a cross-sectional view of an exemplary single-lever stabilizer clamp for use with the stabilizer rail system of FIGS. 10A and 10B, according to an example.

FIG. 12 is a cross-sectional view of an exemplary mouth portion of a stabilizer clamp, according to an example.

FIG. 13 is a cross-sectional view of an exemplary mouth portion of a stabilizer clamp, according to another example.

FIG. 14 is a cross-sectional view of an exemplary single-lever stabilizer clamp including a locking mechanism for use with the stabilizer rail system of FIGS. 10A and 10B, according to an example.

FIG. 15 is a cross-sectional view of an exemplary dual-lever stabilizer clamp for use with the stabilizer rail system of FIGS. 10A and 10B, according to an example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, the term “approximately” means within a specified range relative to a given and/or stated value. For example, the term “approximately” can mean within +/−10% of a given value, within +/−8% of a given value, within +/−5% of a given value, within +/−1% of a given value, etc.

### Introduction to the Disclosed Technology

Described herein are examples of steerable guide catheter apparatus and steerable delivery apparatus (sometimes referred to as a steerable catheter) that can be used to navigate a subject's vasculature to deliver an implantable medical device (for example, a prosthetic heart valve or a docking device), tools, agents, or other therapy to a location within the body of a subject. Examples of procedures in which the guide catheters and steerable catheters are useful include transcatheter aortic procedures, neurological, urological, gynecological, fertility (for example, in vitro fertilization, artificial insemination), laparoscopic, arthroscopic, transesophageal, transvaginal, transvesical, transrectal, and/or procedures including access in any body duct or cavity. As noted above, examples include placing implants, including, for example, prosthetic valves, docking devices, stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

In connection therewith, various systems, apparatus, and methods are described herein that, in some examples, can be used to stabilize and/or enable control of one or more of a guide catheter apparatus or a delivery apparatus during, for example, the foregoing exemplary implantation procedures or other implantation procedures. For example, the guide catheter apparatus can include a first handle disposed at its proximal end. The delivery apparatus can include a second handle disposed at its proximal end. An elongate shaft of the delivery apparatus can extend through a lumen of the guide catheter and the first handle, such that the second handle (of the delivery apparatus) is disposed proximally relative to the first handle. In some examples, a guide catheter can be inserted into the subject and steered to the implantation site via a first handle. In some examples, a delivery apparatus including a docking device retained therein can be navigated through a main lumen of the guide catheter toward a target location for the docking device via a second handle. In some examples, a delivery apparatus including a prosthetic heart valve retained therein can be navigated through a main lumen of the guide catheter toward a target location for the prosthetic heart valve a second handle.

In some examples, the guide catheter apparatus and the delivery apparatus can be used in combination with a stabilizer rail or table system to enable positioning and stabilization of the delivery apparatus handles during an implantation procedure. For example, a portion of each of the first and second handles of the guide catheter apparatus and the delivery apparatus can be attached the stabilizer system or apparatus to enable positioning and stabilization of the handles during implantation procedures. In some examples, the stabilizer system or apparatus can include a rail and one or more stabilizer clamps that can be attached to the rail in manner that enables positioning of the clamp, such as, for example, being slidably attached to the rail. In some examples, the handle of just one of the guide catheter apparatus or the delivery apparatus can be attached to the stabilizer system. In some examples, the handles of both the guide catheter apparatus and the delivery apparatus can be attached to the stabilizer system. In some examples, the handles of both the guide catheter apparatus and the delivery apparatus can be attached in tandem to the stabilizer system in a linear arrangement. For example, the first handle of the guide catheter apparatus can be attached to the rail by a first stabilizer clamp that is distal relative to a main body of the first handle, and the second handle of the delivery apparatus can be attached to the rail by a second stabilizer clamp that is proximal relative the first handle and distal relative to main body of the second handle. The linear or axial arrangement of the handles can allow an elongate shaft of the delivery apparatus to extend through a lumen of the guide catheter and the first handle, and attachment to the rail or table can retain a position and/or enable stable positioning of each of the handles relative to each other and relative to the stabilizer apparatus.

In some examples, the stabilizer clamps can each include a base portion configured to couple with the stabilizer rail or table and a mouth or jaw portion configured to receive and clamp a portion of a handle. For example, the mouth or jaw portion of the stabilizer clamp can be configured to receive a distal cap attached to or integral with a main body housing of the handle. In some examples, the distal end cap and/or other portions of the first handle can be fixed to the guide catheter such that application of a rotational force on the first handle can cause application of torque on the guide catheter. In some examples, the distal end cap and/or other portions of the second handle can be fixed to the elongate shaft such that application of a rotational force on the second handle can cause application of torque on the elongate shaft. In some examples, the mouth or jaw portion of the stabilizer clamp can be configured to enable rotation of the portion of the handle (for example, the distal cap) therein when torque or rotational force is applied to the handle by an operator, and can be further configured to resist or limit rotation of the portion of the handle (for example, the distal cap) therein when no torque or rotational force is applied to the handle by an operator. In some examples, the stabilizer clamp be configured to balance or equalize an amount force required for rotation and application of torque and/or a degree of rotational resistance experienced by the user operating the handle when rotating in first (for example, clockwise) direction and when rotating in a second (for example, counterclockwise) direction. In some examples, a stabilizer clamp is configured such that a ratio of torque resistance for rotation of the handle in a first direction relative to torque resistance for rotation of the handle in a second opposing direction is within a range of 0.8 to 1.2. In some examples, a stabilizer clamp is configured such that a ratio of torque resistance for rotation of the handle in a first direction relative to torque resistance for rotation of the handle in a second opposing direction is approximately 1.0.

In some examples, a stabilizer clamp can include a stationary jaw member and a moveable jaw member. The moveable jaw member can be a lever that rotates over or around a fulcrum on the stationary jaw member or on the base of the stabilizer clamp. The base portion can be from one end (for example, a lower end) of the stabilizer clamp and the stationary jaw member and a moveable jaw member can extend from the base portion. The mouth portion can have an opening at an opposing end (for example, an upper end) of the stabilizer clamp relative to the base portion. The stationary jaw member and the moveable jaw member can each include a curved interior wall, the curved interior walls can cooperatively define the mouth portion. In some examples, the mouth portion can form a generally cylindrical space or void configured to receive a distal cap of a delivery apparatus handle. The moveable jaw member can be configured to be moved between an open position and a closed position for opening and closing of the mouth. One or more spring members can be disposed between the stationary jaw member or base portion and the moveable jaw member or lever, which can bias the moveable jaw member toward to the closed position. The opening to the mouth portion can be wider in the open position of the moveable jaw member to enable insertion (or removal) of the distal cap of the handle, and narrower in the closed position of the moveable jaw member to enable clamping on an exterior surface of the distal cap. In some examples, the spring member can be disposed at an angle in a relative to a longitudinal axis of the stabilizer clamp. In some examples, the spring member is parallel to a longitudinal axis of the stabilizer clamp. In some examples, a minimum torque applied to or required to overcome the clamping force of the stabilizer clamp is at least 21 N-cm. In some examples, a minimum torque required for rotation of the handle in one or more of the first direction or the second opposing direction is in a range of 15 N-cm to 30 N-cm. In some examples, the spring member can have a spring rate of in a range of 5 lbs./in. to 50 lbs./in., such as, for example, 30 lbs./in., which is sufficient to resist axial rotation of a handle when no rotational force (such as application of torque on the handle by an operator) acts upon the handle.

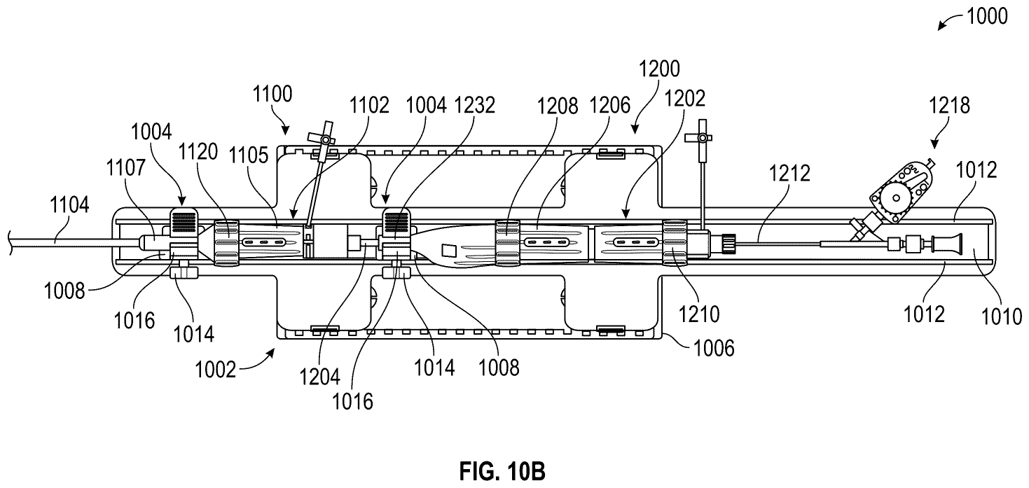

In some examples, such as the example illustrated in FIG. 11, a surface area of the curved interior wall of the moveable jaw member can be less than a surface area of the curved interior wall of the stationary jaw member. In some examples, the curved interior wall of the moveable jaw member includes a reduced or recessed (upper) lip at the opening of the mouth portion. In some examples, when the moveable jaw member is in the closed position, the reduced lip is a greater distance from a vertical axis of the mouth portion than an upper lip of the stationary jaw member. In such examples, the reduced surface area of the curved interior wall of the moveable jaw member at the opening of the mouth can reduce torque resistance in a (first) direction of rotation away from the moveable jaw member (lever) so that a ratio of torque resistance for rotation of the handle in the first direction relative to torque resistance for rotation of the handle in a second opposing direction is within a range of 0.8 to 1.2.

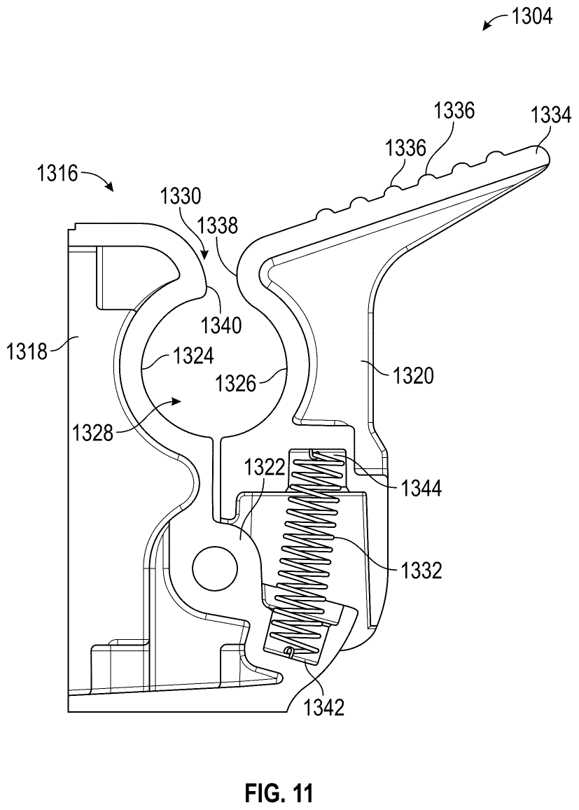

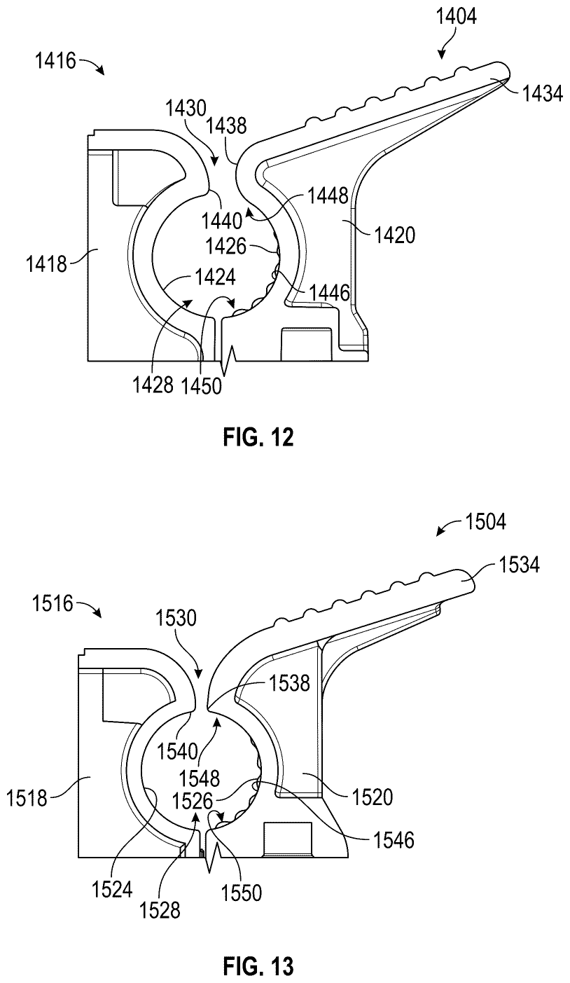

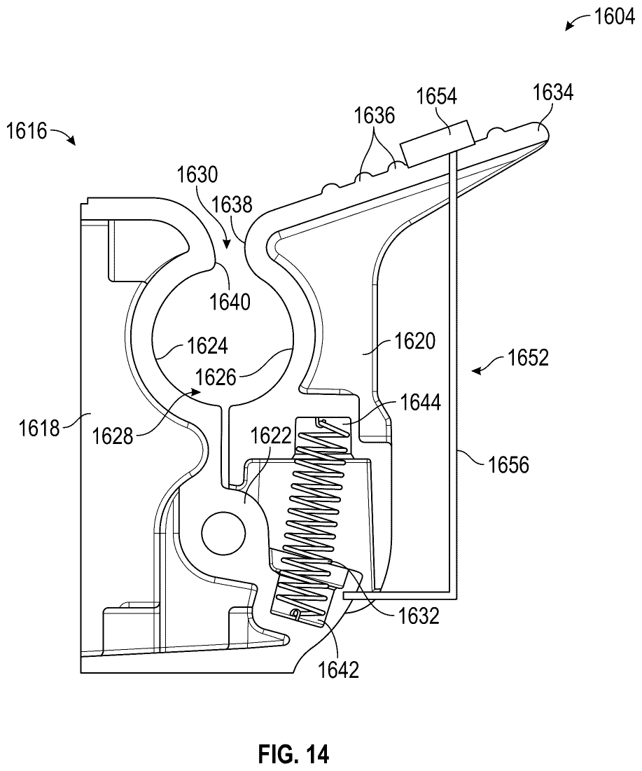

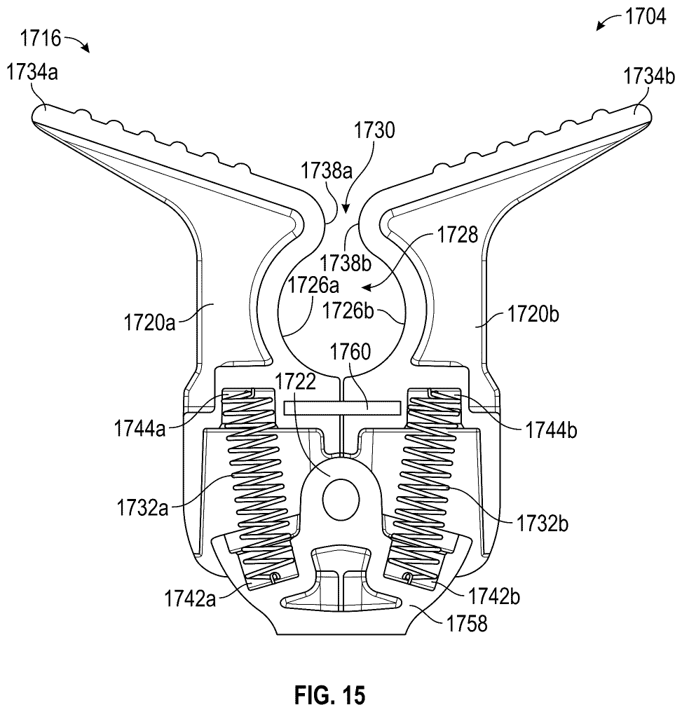

In some examples, a stabilizer clamp can include additional or other features for controlling and/or balancing torque resistance. In some examples, one or more of the curved interior walls of the mouth portion can include a textured surface or a high friction to low friction gradient surface (FIGS. 12 and 13). In some examples, a stabilizer clamp can include a locking mechanism (FIG. 14). In some examples, a stabilizer clamp can include a dual-lever mechanism (FIG. 15).

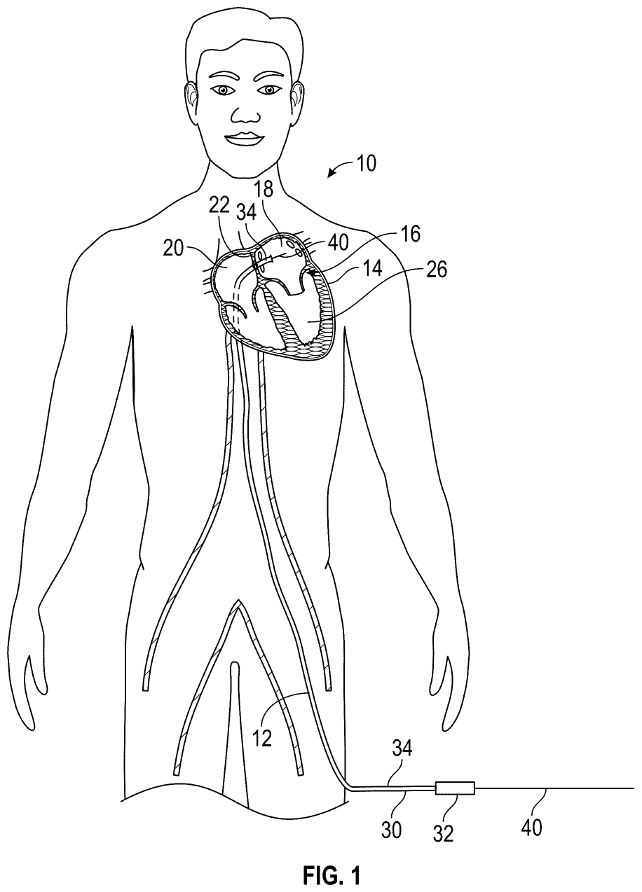

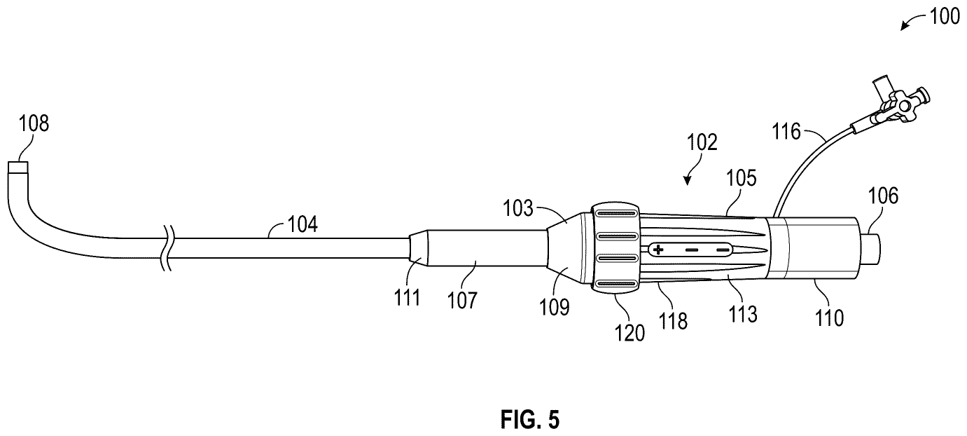

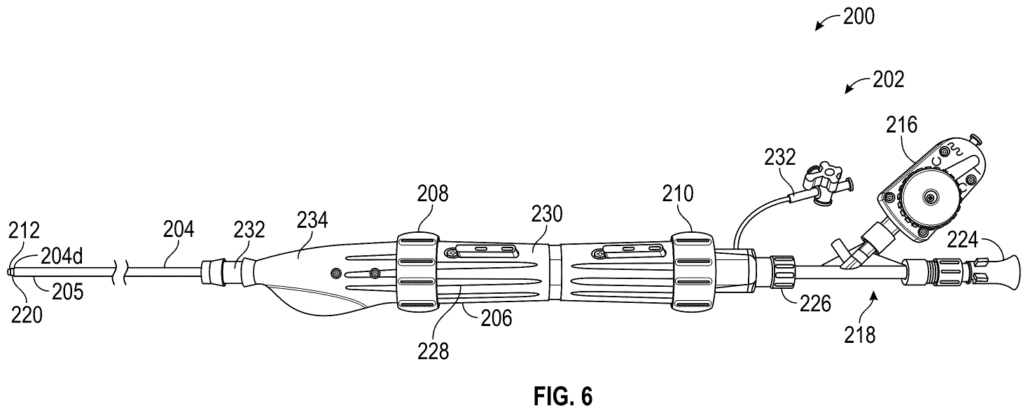

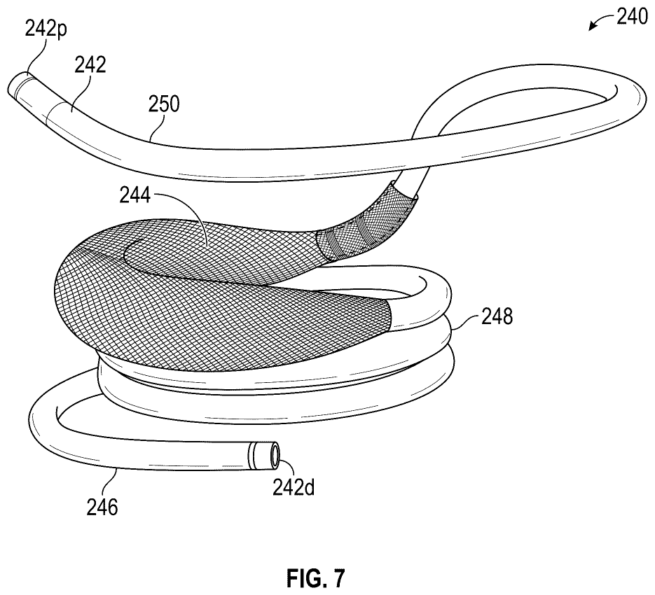

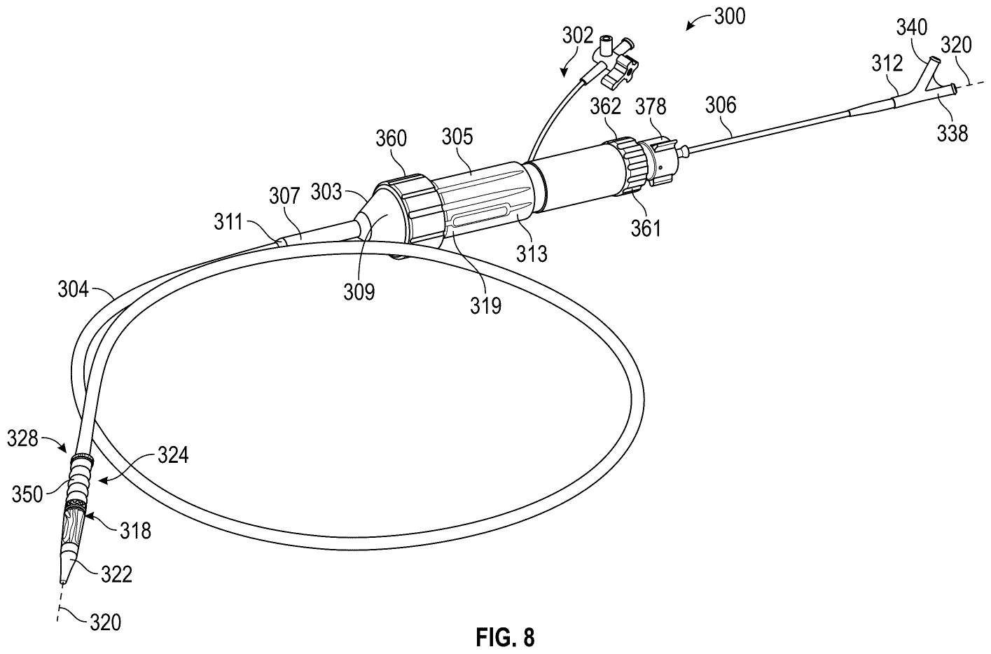

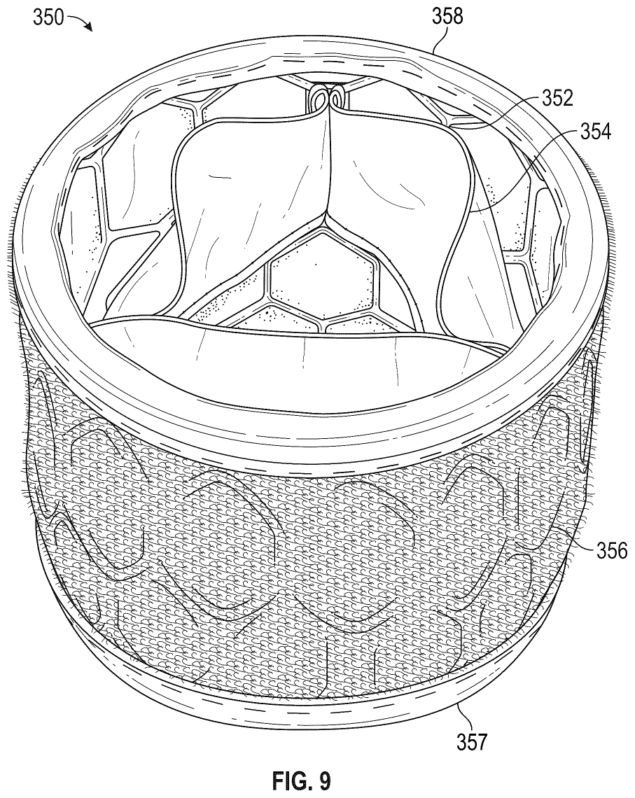

In some examples, the delivery apparatus disclosed herein can be used to introduce one or more delivery shafts, guide catheters, and/or implant catheters into the vasculature of a patient and guide the one or more delivery apparatus at least partially through the vasculature toward a target implantation site. For example, FIGS. 1-4 schematically illustrate an example transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus and then the prosthetic heart valve delivery apparatus is used to deliver a transcatheter prosthetic heart valve inside the docking device. An exemplary guide catheter is shown in more detail in FIG. 5. An exemplary delivery apparatus for delivering a docking device at a native heart valve is shown in FIG. 6, and an example docking device is shown in FIG. 7. An example delivery apparatus for delivering a prosthetic heart valve at a native heart valve is shown in FIG. 8, and an example prosthetic heart valve is shown in FIG. 9.

### Examples of the Disclosed Technology

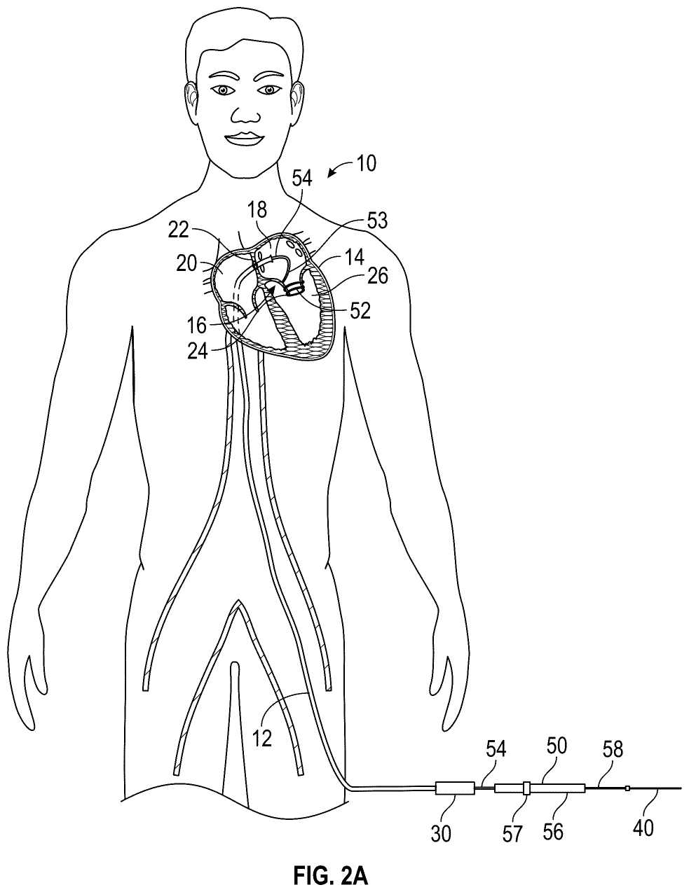

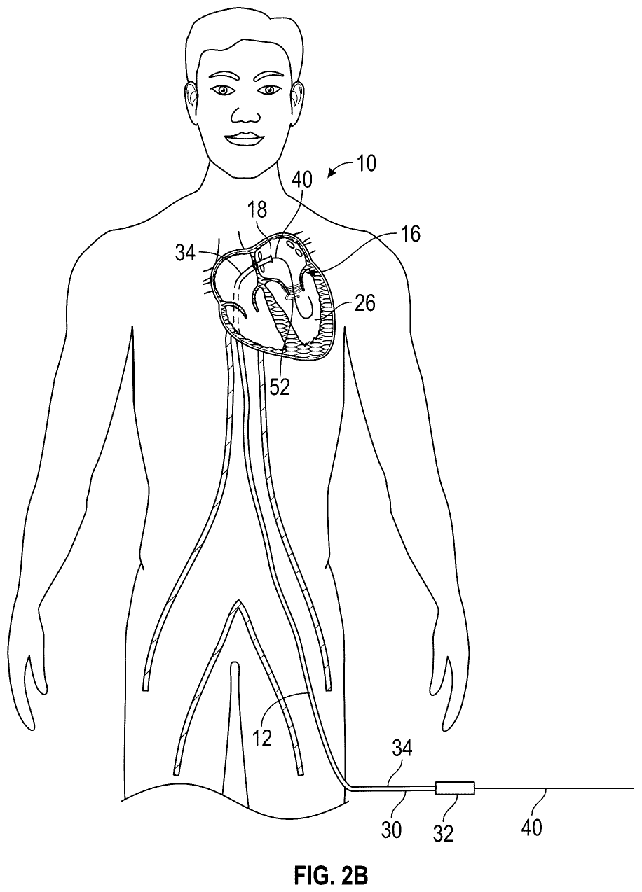

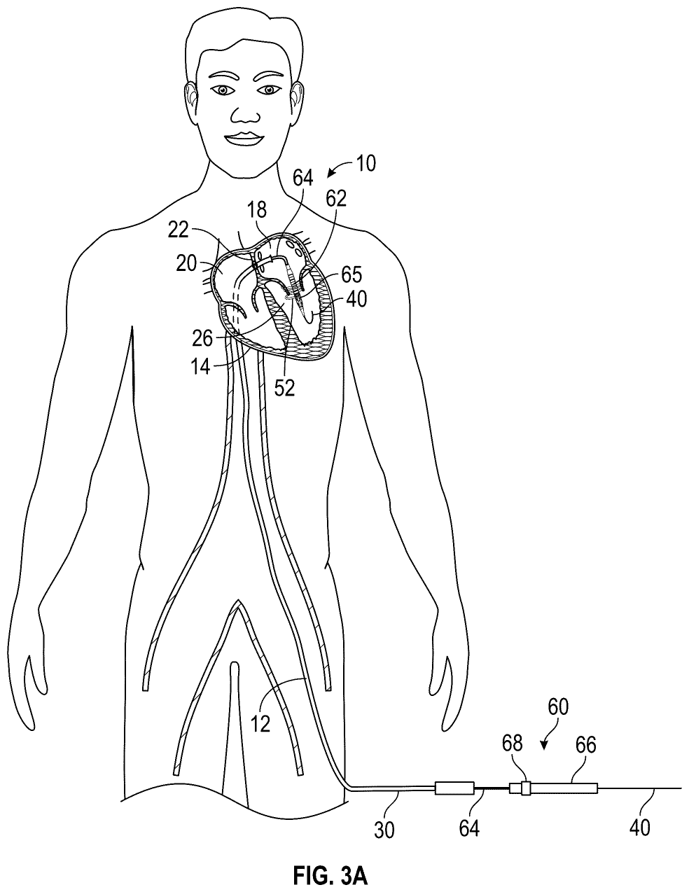

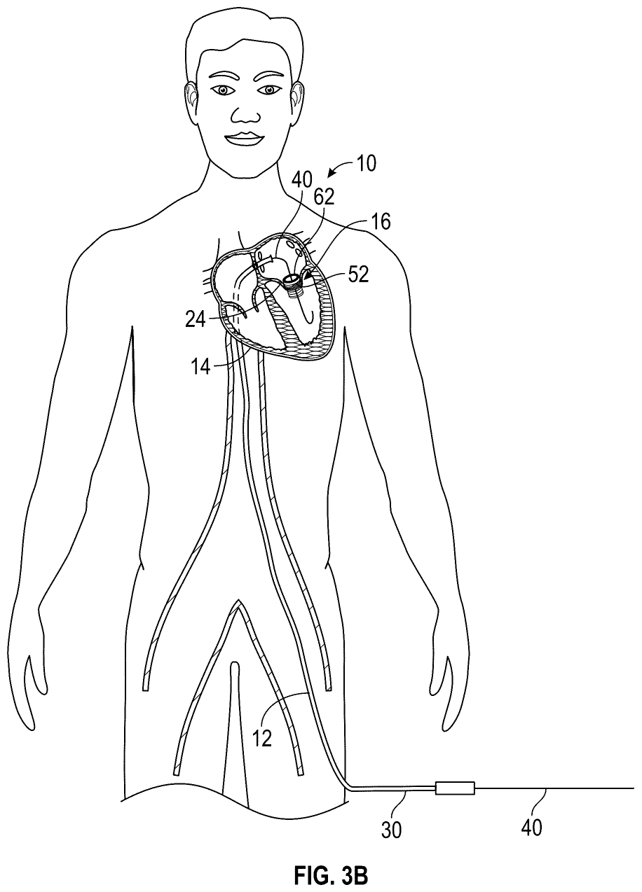

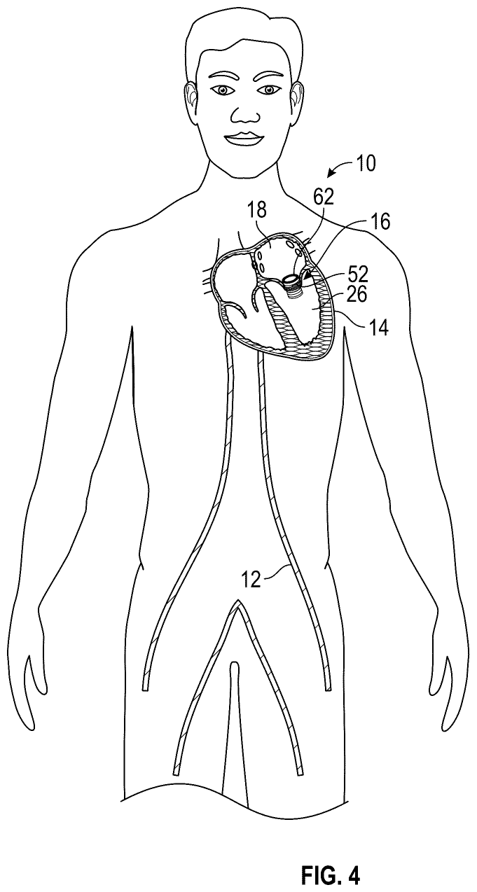

FIGS. 1-4 depict an example of a transcatheter heart valve replacement procedure (for example, a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus). As shown, the heart 14 is illustrated schematically. For example, the anterior leaflet and chordac of the native mitral valve 16 are omitted for illustration purposes, such that only a portion of the posterior leaflet of the native mitral valve 16 is illustrated.

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (for example, the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (for example, native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (for example, blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (for example, the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

Turning now to FIG. 5, an exemplary guide catheter, which is referred to below as a guide sheath 100 (and can also be referred to herein as a “delivery apparatus” or an “introducer device” or an “introducer”) is shown. In some examples, the guide sheath 100 can be used in lieu of or as the guide catheter 30 in a docking device and/or a prosthetic valve implantation procedure, as described above with reference to FIGS. 1-4. The guide sheath 100 can be configured to be inserted into a patient's vasculature and receive an implant catheter or delivery apparatus therein (for example, such as the delivery apparatus 200 of FIG. 6 and/or the delivery apparatus 300 shown in FIG. 8) in order to introduce the implant catheter into the patient's vasculature and at least partially guide the implant catheter therein to a target implantations site. Though the guide sheath 100 is described herein as being used with the delivery apparatus 200, 300, the guide sheath 100 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 100 in the illustrated example comprises a handle assembly 102, an elongated shaft 104 extending distally from the handle assembly 102 along a central longitudinal axis. The shaft 104 can have a main (or primary) lumen that is defined by an inner surface of a wall of the shaft 104. The main lumen can be configured to receive a delivery apparatus therein (such as any of the prosthetic device delivery apparatuses or implant catheters described herein, for example, the delivery apparatus 200, 300). In some examples, the shaft 104 can extend into the handle assembly 102. Further, in some examples, the main lumen can extend through the handle assembly 102 to an inlet port 106 disposed at a proximal end of the handle assembly 102. Thus, in some examples, an inner surface of a wall of a portion of the handle assembly (for example, at the proximal end) can define a proximal portion the main lumen. Thus, the main lumen can extend from the inlet port 106 to a distal end 108 of the shaft 104.

A handle 105 of the handle assembly 102 can be coupled to the shaft 104 such that the handle 105 is configured to transmit torque exerted on the handle to the shaft 104, and thereby cause or result in axial rotation of the shaft 104 when the handle 105 is rotated. The handle assembly 102 can have a housing 113 (also referred to as an “outer housing”) comprising a nose portion 103 and a main body portion 118. The main body portion 118 can be configured to be grasped by a user or operator for application of force thereon to drive rotation of the handle. In some examples, the main body portion 118 of the handle 105 can have a cross-sectional shape (for example, hexagonal, octagonal, etc.) to help facilitate grip of the housing 113.

The nose cone portion 103 can include a frustoconical base 109 attached to the main body portion 118 and a cylindrical cap 107 extending distally from or being positioned over or on a distal end of the base 109. The cylindrical cap 107 can include a frustoconical distal tip 111. In some examples, the cylindrical cap 107 can include a coupler disposed therein that couples or attaches the nose cone 103 (and the handle 105) to the shaft 104. The cap 107 can be configured to prevent movement (for example, axial movement, etc.) of the coupler relative to the nose cone 103 (and the handle 105). In some examples, the cap 107 can comprise an elastomeric material or can have an elastomeric coating. In some examples, the cap 107 can include an interior shape (for example, hexagon, etc.) that is complementary to the shape (for example, hexagon, etc.) of the outer surfaces of the coupler in order to limit or prevent rotation of the cap 107 relative to the coupler and the nose cone 103.

The coupler disposed inside of the cap 103 can be configured to cover (for example, surround) at least a portion of the shaft 104. The coupler can support the shaft 104 as torque is applied to the handle 105. Specifically, the coupler can be connected to the housing 113 (and/or fixed components within the housing) and can transfer torque exerted on the handle 105 (such as, a rotational force applied to the main body portion 118 of the housing 113 of the handle by an operator of the assembly) from the handle 105 to the shaft 104, including to the distal end portion 108 of the shaft 104.

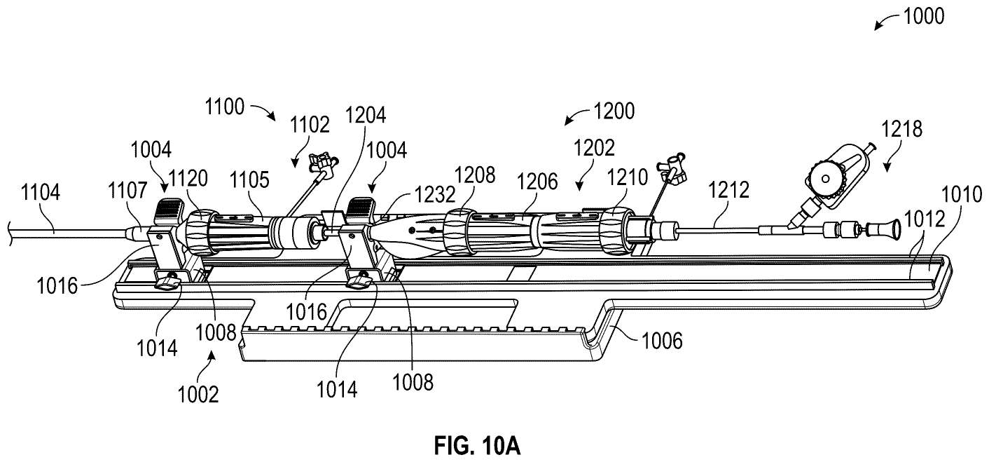

In some examples, the nose cone portion 103 can have a different configuration. For example, the cap 107 can include one or more ridges or grooves, or can have a configuration similar to other caps disclosed herein (such as, the cap 232 that includes include a narrower or recessed central portion having an annular flange or shoulder at each end thereof as shown in FIG. 6 and discussed below). In some examples, the cap 107 is configured to be received by or coupled to a stabilizer clamp, such as the stabilizer clamps shown in FIGS. 10A-15, which are discussed in detail below.

In some examples, the handle assembly 102 can further include a seal housing assembly 110 (which can also be referred to as a “seal stack” and which comprises one or more seals contained therein) within the housing 113. The one or more seals of the seal housing assembly 110 can be configured to fluidly seal the main lumen of the guide sheath 100 from the external environment. For example, the one or more seals of the seal housing assembly 110 can be configured to prevent blood from a patient in which the guide sheath 100 is inserted from exiting the guide sheath 100 and prevent air from the environment from entering the guide sheath 100 (for example, through the inlet port 106). The one or more seals can include a variety of types of seals, such as a duckbill seal, a flapper seal, an umbrella valve, a cross-slit valve, a dome valve, or the like.

The main body portion 118 is disposed adjacent and distal to the seal housing assembly 110. The handle 102 can include a steering mechanism configured to adjust the curvature of the distal end portion of the shaft 104 (as such, the shaft 104 can be referred to as a steerable shaft). In the illustrated example, the handle assembly 102 includes an adjustment member, such as the illustrated rotatable knob 120. In some examples, the handle assembly 102 can include buttons, wheels, and/or other means for controlling and/or actuating one or more components of the guide sheath 100. The main body portion 118 can house internal flex mechanisms of the guide sheath 100 which are operatively coupled to the rotatable knob 120. In some examples, the flex mechanisms, and thus the knob 120, can be operatively coupled to the proximal end portion of a pull wire. The pull wire can extend distally from the handle assembly 102 through the shaft 104 and have a distal end portion affixed to the shaft 104 at or near the distal end 108 of the shaft 104. Rotating the knob 120 can increase or decrease the tension in the pull wire, thereby adjusting the curvature of the distal end portion of the shaft 104. Further details on steering or flex mechanisms for a delivery apparatus can be found in U.S. Pat. No. 9,339,384, which is incorporated by reference herein.

The handle 102 can include a flush port 116 connected to the housing 113, distal to the seal housing assembly 110. In some examples, the flush port 116 is connected to the main body portion 118 of the housing 113. The handle assembly 102 can further include a compressible reservoir disposed within the housing 113. The reservoir can be filled with a fluid have an adjustable fluid volume. The reservoir can be fluidly coupled to the flush port 116 by a channel (or flush lumen).

As introduced above, the guide sheath 100 can be configured to receive a delivery apparatus, such as the delivery apparatus 200 of FIG. 6 and/or the delivery apparatus 300 of FIG. 8, within the main lumen of the guide sheath 100. Prior to inserting the delivery apparatus 200, 300 into the guide sheath (and/or prior to inserting the guide sheath into the vasculature of the patient), the main lumen and reservoir of the guide sheath 100 can be primed or flushed through the flush port 116. For example, fluid can flow through the flush port 116 into the cavity of the reservoir. In some instances, the cavity can be filled with fluid until the wall of the reservoir expands as far as possible and hits a wall of the housing 113 or a wall of the flex mechanisms. Once the reservoir is full (and in its expanded configuration), fluid entering the reservoir from the flush port 116 can continue to flow into the main lumen. In some instances, this process can continue until the main lumen is filled to a desired level. In some examples, the fluid used to fill the reservoir and main lumen is saline or an alternate biocompatible flush fluid.

After positioning the shaft 104 of the guide sheath 100 within the vasculature of a patient, a distal end portion of the delivery apparatus 200, 300 can be inserted into the inlet port 106 of the handle 102 of the guide sheath 100. The distal end portion of the delivery apparatus 200, 300 can then be navigated through the seal housing assembly 110 and into the main lumen of the guide sheath 100 within the handle assembly 102. The delivery apparatus 200, 300 can then continue to be navigated through the main lumen of the shaft 104, toward the implantation site. An exemplary implant delivery assembly or system 400 including the guide sheath 100 and the delivery apparatus 200 is shown in FIGS. 10A and 10B, discussed further below.

Additional examples and details regarding the guide sheath and functions and methods of use thereof are described in U.S. Provisional Patent Application 63/268,322, filed on Feb. 22, 2022, which is incorporated by reference herein.

FIG. 6 illustrates an exemplary delivery apparatus 200 configured to implant a docking device, such as docking device 240 (FIG. 7) described below or other docking devices, to a target implantation site in a patient. For example, the delivery apparatus 200 can be used as the docking device delivery apparatus 50 in a prosthetic valve implantation procedure, as described above with reference to FIG. 2A. The delivery apparatus 200 can also be referred to as a “dock delivery catheter” or “dock delivery system.”

As shown, the delivery apparatus 200 can include a handle assembly 202 and a delivery sheath 204 (also referred to as the “delivery shaft” or “outer shaft” or “outer sheath”) extending distally from the handle assembly 202. The handle assembly 202 can include a handle 206 coupled to the delivery sheath 204 such that the handle 206 is configured to transmit torque exerted on the handle to the delivery sheath 204, and thereby cause or result in axial rotation of the delivery sheath 204 when the handle 206 is rotated. Further, the handle assembly 202 can include one or more knobs, buttons, wheels, and/or other means for controlling and/or actuating one or more components of the delivery apparatus 200. For example, in some examples, as shown in FIG. 6, the handle assembly 202 can include knobs 208 and 210 which can be configured to steer or control flexing of the delivery apparatus 200, such as flexing of the delivery sheath 204 and/or the sleeve shaft 220 described below.

In certain examples, the delivery apparatus 200 can also include a pusher shaft 212 and a sleeve shaft 220, both of which can extend through an inner lumen of the delivery sheath 204 and have respective proximal end portions extending into the handle assembly 202.

A distal end portion (also referred to as “distal section”) of the sleeve shaft 220 can be configured to cover (for example, surround) a docking device 240 (see FIG. 7). For example, the docking device 240 can be retained inside the sleeve shaft 220, which is further retained by a distal end portion 205 of the delivery sheath 204, when navigating through a patient's vasculature.

As noted above, the handle 206 can be coupled to the proximal region of the delivery sheath 204 and can be configured to transmit torque exerted on the handle 206 to the delivery sheath 204. In some examples, a housing 228 of the handle 206 (at a main body portion 230 of the handle) can have a cross-sectional shape (for example, hexagonal, octagonal, etc.) to help facilitate grip of the handle 206.

In some examples, a cap 232 can extend from or be positioned over or on a distal end of a nose cone portion 234 of the handle 206 and can include a coupler disposed therein that couples or attaches the nose cone 234 (and the handle 206) to the delivery sheath 204. The cap 232 can be configured to prevent movement (for example, axial movement, etc.) of the coupler relative to the nose cone 234 (and the handle 206). In some examples, the cap 232 can comprise an elastomeric material. In some examples, the cap 232 can include an interior shape (for example, hexagon, etc.) that is complementary to the shape (for example, hexagon, etc.) of the outer surfaces of the coupler in order to limit or prevent rotation of the cap 232 relative to the coupler and the nose cone 234. In some examples, the cap 232 (as shown in FIG. 6) can include a narrower or recessed central portion having an annular flange or shoulder at each end thereof (formed by a frustoconical proximal end region and a frustoconical distal end region). In some examples, the cap 232 can comprise other shapes including, for example, a cylindrical shape or a cylinder including an annular ridge at the proximal end region and/or the distal end region, etc.

The coupler disposed inside of the cap 232 can be configured to cover (for example, surround) at least a portion of the delivery shaft 204. The coupler can support the delivery shaft 204 as torque is applied to the handle 206. Specifically, the coupler can be connected to the housing 228 (and/or fixed components within the housing) and can transfer torque exerted on the handle 206 (such as, a rotational force applied to the main body portion 230 of the handle by an operator of the assembly) from the housing 228 of the handle 206 to the delivery shaft 204, including to the distal end portion 205 of the delivery shaft 204.

In some examples, the nose cone portion 234 can have a different configuration. For example, the cap 232 can include one or more ridges or grooves, or can have a configuration similar to other caps disclosed herein (such as, the cylindrical configuration of the cap 107 shown in FIG. 5 and discussed above). In some examples, the cap 232 is configured to be received by or coupled to a stabilizer clamp, such as the stabilizer clamps shown in FIGS. 10A-15, which are discussed in detail below.

The distal end portion 205 of the delivery sheath 204 can be configured to be steerable. In one example, by rotating a knob (for example, 208 or 210) on the handle 206, a curvature of the distal end portion 205 can be adjusted so that the distal end portion 205 of the delivery sheath 204 can be oriented in a desired angle. For example, to implant the docking device 240 at the native mitral valve location, the distal end portion 205 of the delivery sheath 204 can be steered in the left atrium so that at least a portion of the sleeve shaft 220 and the docking device 240 retained therein can extend through the native mitral valve annulus at a location adjacent the posteromedial commissure.

In some examples, the pusher shaft 212 and the sleeve shaft 220 can be coaxial with one another, at least within the delivery sheath 204. In some examples, the delivery sheath 204 can be configured to be axially movable relative to the sleeve shaft 220 and the pusher shaft 212. As described further below, a distal end of the pusher shaft 212 can be inserted into a lumen of the sleeve shaft 220 and press against the proximal end of the docking device 240 retained inside the sleeve shaft 220.

After reaching a target implantation site, the docking device 240 can be deployed from the delivery sheath 204 by manipulating the pusher shaft 212 and sleeve shaft 220 using a hub assembly 218, as described further below. For example, by pushing the pusher shaft 212 in the distal direction while holding the delivery sheath 204 in place or retracting the delivery sheath 204 in the proximal direction while holding the pusher shaft 212 in place, or pushing the pusher shaft 212 in the distal direction while simultaneously retracting the delivery sheath 204 in the proximal direction, the docking device 240 can be pushed out of a distal end 204d of the delivery sheath 204, thus changing from a delivery configuration of the docking device 240 (for example, an elongated configuration) to a deployed, coiled configuration (see FIG. 7). In certain examples, the pusher shaft 212 and the sleeve shaft 220 can be actuated independently of each other.

During delivery, the docking device 240 can be coupled to the delivery apparatus 200 via a release suture (or other retrieval line comprising a string, yarn, or other material that can be configured to be tied around the docking device 240 and cut for removal) that extends through the pusher shaft 212. In one specific example, a release suture can extend through the delivery apparatus 200, for example, through an inner lumen of the pusher shaft 212, to a suture lock assembly 216 of the delivery apparatus 200.

The handle assembly 202 can further include the hub assembly 218 to which the suture lock assembly 216 and a sleeve handle 224 are attached. The hub assembly 218 can be configured to independently control the pusher shaft 212 and the sleeve shaft 220 while the sleeve handle 224 can control an axial position of the sleeve shaft 220 relative to the pusher shaft 212. In this way, operation of the various components of the handle assembly 202 can actuate and control operation of the components arranged within the delivery sheath 204. In some examples, the hub assembly 218 can be coupled to the handle 206 via a connector 226.

The handle assembly 202 can further include one or more flush ports (for example, flush port 232 is shown in FIG. 6) to supply flush fluid to one or more lumens arranged within the delivery apparatus 200 (for example, annular lumens arranged between coaxial components of the delivery apparatus 200).

Further details on delivery apparatus/catheters/systems (including various examples of the handle assembly) that are configured to deliver a docking device to a target implantation site can be found in International Application No. PCT/US2020/036577, in U.S. Patent Publication Nos. 2018/0318079 and 2018/0263764, and in U.S. Provisional Patent Application No. 63/363,162 (filed on Apr. 18, 2022), which are each incorporated by reference herein.

FIG. 7 illustrates the docking device 240, according to one example. The docking device 240 can, for example, be used as the docking device 52 in a prosthetic valve implantation procedure, as described above with reference to FIGS. 1-4. As depicted in FIG. 7, the docking device 240 in its deployed configuration can be configured to receive and secure a prosthetic valve within the docking device, thereby securing the prosthetic valve at the native valve annulus.

The docking device 240 can comprise a coil member 242 and a guard member 244 covering at least a portion of the coil member 242. In some examples, the coil member 242 can include a shape memory material (for example, nickel titanium alloy or “Nitinol”) such that the docking device 240 (and the coil member 242) can move from a substantially linear configuration (or delivery configuration) when disposed within the delivery sheath 204 of the delivery apparatus 200 to a coiled or helical, deployed configuration after being removed from the delivery sheath 204.

The coil member 242 has a proximal end 242p and a distal end 242d (which also respectively define the proximal and distal ends of the docking device 240). When being disposed within the delivery sheath 204 (for example, during delivery of the docking device 240 into the vasculature of a patient), a body of the coil member 242 between the proximal end 242p and distal end 242d can form a generally straight or linear delivery configuration (that is, without any coiled or looped portions, but can be flexed or bent) so as to maintain a small radial profile when moving through a patient's vasculature. After being removed from the delivery sheath 204 and deployed at an implant position, the coil member 242 can move from the delivery configuration to the helical deployed configuration and wrap around native tissue adjacent the implant position. For example, when implanting the docking device at the location of a native valve, the coil member 242 can be configured to surround native leaflets of the native valve (and the chordae tendineae that connects native leaflets to adjacent papillary muscles, if present).

The docking device 240 can be releasably coupled to the delivery apparatus 200. For example, in certain examples, the docking device 240 can be coupled to a delivery apparatus (as described above) via a release suture that can be configured to be tied to the docking device 240 and cut for removal.

As shown in FIG. 7, the coil member 242 in the deployed configuration can include a leading turn 246 (or “leading coil”), a central region 248, and a stabilization turn 250 (or “stabilization coil”) around a central longitudinal axis. The central region 248 can possess one or more helical turns having substantially equal inner diameters. The leading turn 246 can extend from a distal end of the central region 248 and has a diameter greater than the diameter of the central region 248, in the illustrated example. The stabilization turn 250 can extend from a proximal end of the central region 248 and has a diameter greater than the diameter of the central region 248, in the illustrated example. In some examples, the stabilization turn 250 can be omitted from the coil member 242, for example, when a retention member is used to stabilize the positioning of the docking device 240 relative to the native anatomy during an implant procedure. Alternatively, the stabilization turn 250 can have a diameter that is equal, approximately equal, or less than the diameter of the central region 248 (as opposed to larger), and/or the stabilization turn can comprise less of a full turn than depicted in FIG. 7.

Further details of exemplary docking devices and variants thereof are described in International Application No. PCT/US2021/056150, which is incorporated by reference herein.

FIG. 8 illustrates an exemplary prosthetic heart valve delivery apparatus 300 (which can also be referred to here as an “implant catheter”) that can be used to implant an expandable prosthetic heart valve (such as, an expandable prosthetic heart valve 350 shown in FIG. 9 or other prosthetic heart valves). In some examples, the delivery apparatus 300 is specifically adapted for use in introducing a prosthetic heart valve into a heart. For example, the delivery apparatus 300 can be used as the prosthetic heart valve delivery apparatus 60 in a prosthetic valve implantation procedure, as described above with reference to FIG. 3A.

As shown, the delivery apparatus 300 can be a balloon catheter including a handle assembly 302 and a steerable, outer shaft 304 (also referred to as the “delivery shaft” or “outer shaft” or “outer sheath”) extending distally from the handle assembly 302. The handle assembly 202 can include a handle 305 coupled to the outer shaft 304 such that the handle 305 is configured to transmit torque exerted on the handle to the outer shaft 304, and thereby cause or result in axial rotation of the outer shaft 304 when the handle 305 is rotated. Further, in some examples, the handle assembly 302 can include one or more knobs, buttons, wheels, and/or other means for controlling and/or actuating one or more components of the delivery apparatus 300. For example, as shown in FIG. 8, the handle assembly 302 can include knobs 360 and 362 which can be configured to steer or control flexing of the delivery apparatus 300, such as flexing the outer shaft 304 and/or an intermediate shaft 306 described below.

The delivery apparatus 300 can further comprise an intermediate shaft 306 (which also may be referred to as a balloon shaft) that extends proximally and distally from the handle assembly 302, where the portion extending distally from the handle 302 extends coaxially through the outer shaft 304. In some examples, the delivery apparatus 300 can further comprise an inner shaft extending distally from the handle assembly 302 coaxially through the intermediate shaft 306 and the outer shaft 304 and proximally from the handle assembly 302 coaxially through the intermediate shaft.

The outer shaft 304 and the intermediate shaft 306 can be configured to translate (for example, move) longitudinally, along a central longitudinal axis 320 of the delivery apparatus 300, relative to one another to facilitate delivery and positioning of a prosthetic valve at an implantation site in a patient's body.

The intermediate shaft 306 can include a proximal end portion that extends proximally from a proximal end of the handle 302 to an adaptor 312. The adaptor 312 can include a first port 338 configured to receive a guidewire therethrough and a second port 340 configured to receive fluid (for example, inflation fluid) from a fluid source. The second port 340 can be fluidly coupled to an inner lumen of the intermediate shaft 306.

In some examples, the intermediate shaft 306 can further include a distal end portion that extends distally beyond a distal end of the outer shaft 304 when a distal end of the outer shaft 304 is positioned away from an inflatable balloon 318 of the delivery apparatus 300. In some examples, a distal end portion of the inner shaft can extend distally beyond the distal end portion of the intermediate shaft 306 toward or to a nose cone 322 at a distal end of the delivery apparatus 300.

In some examples, a distal end of the balloon 318 can be coupled to a distal end of the delivery apparatus 300, such as being coupled to the nose cone 322 (as shown in FIG. 8), or to an alternate component at the distal end of the delivery apparatus 300 (for example, a distal shoulder). An intermediate portion of the balloon 318 can overlay a valve mounting portion 324 of a distal end portion of the delivery apparatus 300 and a distal end portion of the balloon 318 can overly a distal shoulder of the delivery apparatus 300. As shown in FIG. 8, a prosthetic heart valve 350 can be mounted around the balloon 318, at the valve mounting portion 324 of the delivery apparatus 300, in a radially compressed state. The prosthetic heart valve 350 can be configured to be radially expanded by inflation of the balloon 318 at a native valve annulus, as described above with reference to FIG. 3A.

A balloon shoulder assembly of the delivery apparatus 300, which includes the distal shoulder, is configured to maintain the prosthetic heart valve 350 (or other medical device) at a fixed position on the balloon 318 during delivery through the patient's vasculature.

The outer shaft 304 can include a distal tip portion 328 mounted on its distal end. In some examples, the outer shaft 304 and the intermediate shaft 306 can be translated axially relative to one another to position the distal tip portion 328 adjacent to a proximal end of the valve mounting portion 324, when the prosthetic valve 350 is mounted in the radially compressed state on the valve mounting portion 324 (as shown in FIG. 8) and during delivery of the prosthetic valve to the target implantation site. As such, the distal tip portion 328 can be configured to resist movement of the prosthetic valve 350 relative to the balloon 318 proximally, in the axial direction, relative to the balloon 318, when the distal tip portion 328 is arranged adjacent to a proximal side of the valve mounting portion 324.

An annular space can be defined between an outer surface of the inner shaft and an inner surface of the intermediate shaft 306 and can be configured to receive fluid from a fluid source via the second port 340 of the adaptor 312. The annular space can be fluidly coupled to a fluid passageway formed between the outer surface of the distal end portion of the inner shaft and an inner surface of the balloon 318. As such, fluid from the fluid source can flow to the fluid passageway from the annular space to inflate the balloon 318 and radially expand and deploy the prosthetic valve 350.

An inner lumen of the inner shaft can be configured to receive a guidewire therethrough, for navigating the distal end portion of the delivery apparatus 300 to the target implantation site.

As noted above, the handle 305 can be coupled to the proximal region of the outer shaft 304 and can be configured to transmit torque exerted on the handle 305 to the outer shaft 304. The handle assembly 302 can have a housing 313 (also referred to as an “outer housing”) comprising a nose portion 303 and a main body portion 319. The main body portion 319 can be configured to be grasped by a user or operator for application of force thereon to drive rotation of the handle. In some examples, the main body portion 319 of the handle 305 can have a cross-sectional shape (for example, hexagonal, octagonal, etc.) to help facilitate grip of the housing 313.

The nose cone portion 303 can include a frustoconical base 309 attached to the main body portion 319 and a cylindrical cap 307 extending distally from or being positioned over or on a distal end of the base 309. The cylindrical cap 307 can include a frustoconical distal tip 311. In some examples, the cylindrical cap 307 can include a coupler disposed therein that couples or attaches the nose cone 303 (and the handle 305) to the shaft 304. The cap 307 can be configured to prevent movement (for example, axial movement, etc.) of the coupler relative to the nose cone 303 (and the handle 305). In some examples, the cap 307 can comprise an elastomeric material or can have an elastomeric coating. In some examples, the cap 307 can include an interior shape (for example, hexagon, etc.) that is complementary to the shape (for example, hexagon, etc.) of the outer surfaces of the coupler in order to limit or prevent rotation of the cap 307 relative to the coupler and the nose cone 303.

The coupler disposed inside of the cap 303 can be configured to cover (for example, surround) at least a portion of the shaft 304. The coupler can support the shaft 304 as torque is applied to the handle 305. Specifically, the coupler can be connected to the housing 313 (and/or fixed components within the housing) and can transfer torque exerted on the handle 305 (such as, a rotational force applied to the main body portion 319 of the housing 313 of the handle by an operator of the assembly) from the handle 305 to the outer shaft 304, including to a distal end portion of the outer shaft 304 (including, for example, the valve mounting portion 324).

In some examples, the nose cone portion 303 can have a different configuration. For example, the cap 307 can include one or more ridges or grooves, or can have a configuration similar to other caps disclosed herein (such as, the cap 232 that includes include a narrower or recessed central portion having an annular flange or shoulder at each end thereof as shown in FIG. 6 and discussed above). In some examples, the cap 307 is configured to be received by or coupled to a stabilizer clamp, such as the stabilizer clamps shown in FIGS. 10A-15, which are discussed in detail below.

The handle assembly 302 can further include a steering mechanism configured to adjust the curvature of the distal end portion of the delivery apparatus 300. In the illustrated example, for example, the handle assembly 302 includes an adjustment member, such as a rotatable knob 360, which in turn is operatively coupled to the proximal end portion of a pull wire. The pull wire can extend distally from the handle assembly 302 through the outer shaft 304 and has a distal end portion affixed to the outer shaft 304 at or near the distal end of the outer shaft 304. Rotating the knob 360 can increase or decrease the tension in the pull wire, thereby adjusting the curvature of the distal end portion of the delivery apparatus 300. Further details on steering or flex mechanisms for the delivery apparatus can be found in U.S. Pat. No. 9,339,384, previously incorporated by reference herein.

The handle assembly 302 can further include an adjustment mechanism 361 including an adjustment member, such as a rotatable knob 362, and an associated locking mechanism including another adjustment member, configured as, for example, a rotatable knob 378. The adjustment mechanism 361 can be configured to adjust the axial position of the intermediate shaft 306 relative to the outer shaft 304 (for example, for fine positioning at the implantation site).

Prosthetic valves disclosed herein (for example, the prosthetic heart valve 350, the prosthetic heart valve 62, etc.) can be radially compressible and expandable between a radially compressed state and a radially expanded state. Thus, the prosthetic valves can be crimped on or retained by an implant delivery apparatus (for example, the delivery apparatus 300, the delivery apparatus 60, etc.) in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures.

FIG. 9 illustrates the exemplary prosthetic valve 350 in a radially expanded position or state. The prosthetic valve 350 can be used as the prosthetic heart valve 62 in a prosthetic valve implantation procedure, as described above with reference to FIGS. 1-4. Any of the prosthetic valves disclosed herein can be adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device (for example, the docking device 240 of FIG. 7 or another docking/anchoring device) that is implanted within a native heart valve or a vessel. For example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

As shown in FIG. 7, the prosthetic valve 350 can include a frame 352 and a plurality of leaflets 354 can be situated at least partially within the frame 352. The prosthetic valve 350 can also include an outer covering 356 situated about the frame 352. As shown in FIG. 7, the prosthetic valve 350 includes an inflow end 357 and an outflow end 358. The terms “inflow” and “outflow” are related to the normal direction of blood flow (for example, antegrade blood flow) through the prosthetic valve 350. For example, the leaflets 354 can allow blood flow through the valve 350 in a direction from the inflow end 357 to the outflow end 358 and prevent the reverse flow (for example, prevent flow in a direction from the outflow end 358 to the inflow end 357).

The frame 352 can be made of any of various suitable plastically-expandable materials (for example, stainless steel, etc.) or self-expanding materials (for example, Nitinol) as known in the art. When constructed of a plastically-expandable material, the frame 352 (and thus the valve 350) can be crimped to a radially compressed state on a delivery catheter and then expanded inside a patient by an inflatable balloon or equivalent expansion mechanism. When constructed of a self-expandable material, the frame 352 (and thus the valve 350) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 352) include, metal alloys, polymers, or combinations thereof. Example metal alloys can comprise one or more of the following: nickel, cobalt, chromium, molybdenum, titanium, or other biocompatible metal. In some examples, the frame 352 can comprise stainless steel. In some examples, the frame 352 can comprise cobalt-chromium. In some examples, the frame 352 can comprise nickel-cobalt-chromium. In some examples, the frame 352 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Further details of the prosthetic heart valve and its variants are described in U.S. Pat. No. 11,185,406, which is incorporated by reference herein.

As noted above, the delivery apparatus 200 and/or the delivery apparatus 300 can be introduced into a patient's vasculature via a guide catheter, such as the guide catheter 100 of FIG. 5. For example, to introduce the delivery apparatus 200 and/or 300 (or an alternate implant catheter or delivery apparatus) into the vasculature of a patient, the shaft 104 of the guide catheter 100 can first be inserted into the patient's vasculature and navigated through the vasculature toward a target implantation site for a medical device or implant. The handle assembly 102 of the guide catheter 100 remains outside the patient and can be accessed and/or controlled by a user (for example, a physician). The distal end portion 205 of the delivery apparatus 200 and/or the distal end portion of the delivery apparatus 300 can then be inserted into the main inlet port 130 of the handle assembly 102 of the guide catheter 100 and pushed through the main lumen 112 of the shaft 104, toward the implantation site such as in, for example, the mitral valve replacement procedure described above in connection with FIGS. 1-4).

FIGS. 10A and 10B illustrate an example delivery system 1000 including a guide catheter 1100 (which can be similar to one or more of the guide catheters 30 or 100), a docking device delivery apparatus 1200 (which can be similar to one or more of the delivery apparatus 50 or 200), and a stabilizer apparatus 1002 (also referred to herein as a “stabilizing tower” or “stabilizing device” or “stabilizer system”). The delivery system 1000 can be used in a transcatheter heart valve replacement procedure, for example, as described above with reference to FIGS. 1-4. Specifically, the delivery system 1000 depicted in FIGS. 10A and 10B can be used during the second stage of the procedure described above with reference to FIG. 2A.

As shown in FIGS. 10A and 10B, the guide catheter 1100 can be coupled to the stabilizer apparatus 1002 to stabilize a position of the guide catheter 1100 during and/or after insertion into a subject and navigation or steering to an implantation site via its handle assembly 1102. The delivery apparatus 1200 can be inserted through the guide catheter 1100. Specifically, a delivery shaft 1204 of the delivery apparatus 1200 can be advanced through a central lumen of the guide catheter 1100 (for example, through a central lumen extending through the handle assembly 1102 and an elongate shaft 1104 of the guide catheter 1100). A handle assembly 1202 of the delivery apparatus 1200 includes a hub or pusher assembly 1218, and a shaft 1212 of the hub assembly 1218 can be inserted through a central lumen of the delivery apparatus 1200 and extend distally through the handle assembly 1202 into the delivery shaft 1204. As described above with reference to the hub assembly 218, the hub assembly 1218 can be configured to selectively allow movement (for example, axial and/or rotational movement, etc.) of a pusher shaft relative to the handle assembly 1202 and the delivery shaft 1204.

The guide catheter 1100 and the delivery apparatus 1200 can be coupled to the stabilizer apparatus 1002 which can support and stabilize the guide catheter 1100 and the delivery apparatus 1200 (for example, during the implantation procedure, etc.). The stabilizer apparatus 1002 can include stabilizer clamps 1004 (which can be, for example, clips, clamps, braces, etc.) that can be configured to hold or grip portions of the guide catheter 1100 and the delivery apparatus 1200 and retain positions of the guide catheter 1100 and the delivery apparatus 1200 relative to the stabilizer apparatus 1002 and/or relative to each other.

In some examples, a base portion 1008 of each of the stabilizer clamps 1004 can be slidably coupled to a stabilizer rail or table 1006 and can be relocated or repositioned on the stabilizer rail 1006 in an axial direction. In some examples, the stabilizer rail 1006 includes a linear track 1010 having risers or side walls 1012 on opposing sides of the track 1010. In some examples each rise or side wall 1012 incudes a base, a web, and a head or overhang. In some examples, the base portion 1008 can include grooves on opposing sides of the base portion that are configured to receive the head or overhang of the risers or side walls 1012 and enable the slidable coupling of the stabilizer clamps 1004 to the stabilizer rail 1006.

In some examples, the base portion 1008 can further include a locking mechanism 1014 that can releasably fix a position of the base portion 1008 relative to the stabilizer rail 1006. For example, the locking mechanism 1014 can be a compression locking mechanism that can be actuated by a knob, winding mechanism, or other actuator and can be configured to close around and/or grip a portion of the stabilizer rail 1006, such as, for example, the risers or side walls 1012 or a bottom surface of the stabilizer rail 1006. In some examples, the base portion 1008 can include a silicone layer (for example, a silicone brake) on one or more surfaces, such as, for example, a bottom surface of the base portion. The silicone brake can engage with one or more surfaces of the stabilizer rail 1006, for example, a top surface of a track portion of the stabilizer rail, via actuation of the knob, winding mechanism, or other actuator. The friction between the silicone brake and the surface of the stabilizer rail can lock an axial position of stabilizer clamp 1004 relative to the stabilizer rail 1006. In some examples, one or more surfaces of the stabilizer rail can include ridges and/or protrusions perpendicular to the direction travel of the stabilizer clamp (for example, perpendicular to an axial direction of travel) and/or a high friction texture or coating to increase friction between the brake and the surface of the stabilizer rail. In some examples, the locking mechanism 1014 can be configured to widen the base so that it is braced between the interior surfaces of the risers or side walls 1012. In some examples, the locking mechanism can have additional and/or alternate features and configurations.

It will be appreciated that the positions of the stabilizer clamps 1004 can be adjusted to accommodate the lengths of the handle assemblies 1102 and 1202 and/or the desired relative positions of the guide catheter 1100 and the delivery apparatus 1200 so that they can be affixed in tandem to the stabilizer apparatus 1002 with appropriate spacing for insertion of the delivery sheath or shaft 1204 into the central lumen of the guide catheter 1100.

In some examples, as depicted in FIGS. 10A and 10B, a jaw or mouth portion 1016 of each the stabilizer clamps 1004 can extend upward from the base portion 1008 and can be configured to engage with a portion of the handle assembly 1102 or 1202. For example, similar to the cap 107, a cap 1107 of the handle assembly 1102 can be fixed relative to its handle 1105 (a portion of the assembly configured to be grasped by a user) and can include a coupler for fixing the handle assembly 1102 to an elongate shaft 1104. The cap 1107 can be received within or clamped within the jaw or mouth portion 1016 of one of the stabilizer clamps 1004 to couple the handle assembly 1102 to the stabilizer apparatus 1002. In another example, similar to the cap 232, a cap 1232 of the handle assembly 1202 can be fixed relative to its handle 1206 (configured to be grasped by a user) and can include a coupler for fixing the handle assembly 1202 to the delivery shaft 1104. The cap 1232 can be received within or clamped within the jaw or mouth portion 1016 of one of the stabilizer clamps 1004 to couple the handle assembly 1102 to the stabilizer apparatus 1002.

In some examples, torque may be exerted on the handle 1105 of the guide catheter 1100 while the cap 1107 is coupled to the stabilizer clamp 1004, and the torque can be transferred to the shaft 1104. In some examples, one or more actuators, such as a knob 1120, of the handle assembly 1102 can be rotated while the cap 1107 is coupled to the stabilizer clamp 1004 and the handle 1105 remains stationary (or where movement of the handle is limited). In some examples, torque may be exerted on the handle 1206 of the delivery apparatus 1200 while the cap 1232 is coupled to the stabilizer clamp 1004, and the torque can be transferred to the delivery sheath 1204. In some examples, one or more actuators, such as knobs 1208 and/or 1210, of the handle assembly 1202 can be rotated while the cap 1232 is coupled to the stabilizer clamp 1004 and the handle 1206 remains stationary (or where movement of the handle is limited).

Accordingly, the stabilizer clamps can be configured to (i) retain an axial position of the handle assemblies 1102 and 1202 relative to each other and relative to the track 1010 of the stabilizer apparatus 1002, (ii) enable axial rotation of the caps 1107 and 1232 retained within the mouth or jaw portion 1016 when torque is exerted on the respective handles 1105 and 1206 of the handle assemblies, (iii) resist axial rotation of the respective handles 1105 and 1206 of the handle assemblies when no torque is exerted on the handle, including when an actuator (such as, the knobs 1120, 1208 and/or 1210) on the handle assembly is rotated, and/or (iv) maintain a position of and/or resist rotation of the caps 1107 and 1232 when torque is built up at a distal end portion of the respective delivery system (that is, the guide catheter 1100 and/or the delivery apparatus 1200).

During operation (for example, steering, navigating, positioning, flexing, etc.) of the guide catheter 1100 and the delivery apparatus 1200, the respective handles 1105 and 1206 thereof can be rotated by an operator in each of a clockwise direction and a counterclockwise direction to adjust the corresponding shaft (that is, the elongate shaft 1104 and the delivery sheath 1204). In some examples, it is desirable for the rotation of the handle in each of the clockwise direction and the counterclockwise direction to require approximately the same amount of force to be applied to the handle. In other words, torque resistance for rotation of the handle in each of the clockwise direction and the counterclockwise direction should be approximately equal such that the operator does not perceive a difference while operating the guide catheter 1100 and/or the delivery apparatus 1200. In some examples, the stabilizer clamps can be configured to balance and/or equalize torque resistance for rotation of the handles in a first direction and a second opposing direction. For example, a stabilizer clamp can be configured such that a ratio of torque resistance for rotation of a handle in a first direction (for example, a clockwise direction) relative to torque resistance for rotation of the handle in a second opposing direction (for example, a counterclockwise direction) can be within a range of 0.8 to 1.2, such as being within a range of 0.9 to 1.1 or being approximately 1.0.

Further, positions of the respective handles 1105 and 1206 should be maintained when the operator stops application of rotational force or torque thereon or otherwise does not apply any rotational force on the handle. Thus, in some examples, the stabilizer clamp can apply a sufficient and/or a minimum compression force onto the handle to limit and/or resist rotational movement of the handle when no rotational force acts upon the handle (such as, where no torque applied to the handle by an operator). In some examples, the stabilizer clamp can apply a sufficient and/or a minimum compression force onto the handle to limit and/or resist rotational movement of the handle when torque is applied to a distal portion of the respective delivery system (that is, the guide catheter 1100 and/or the delivery apparatus 1200) by the patient's body and/or vasculature. In some examples, the stabilizer clamp can apply a sufficient and/or a minimum compression force onto the handle to limit and/or resist rotational movement of the handle such that torque that is built up at a distal portion of the respective delivery system (that is, the guide catheter 1100 and/or the delivery apparatus 1200) can be maintained while the distal portion is within the patient's body and/or vasculature. For example, the stabilizer clamp can include one or more spring members configured to bias the mouth or jaw portion towards a closed position. In some examples, the spring members can have a spring rate of in a range of 5 lbs./in. to 50 lbs./in., such as, for example, 30 lbs./in. In some examples, a minimum torque required to overcome the clamping force is in a range of 15 N-cm to 30 N-cm, such as, for example, 18 N-cm to 25 N-cm. In some examples, a minimum torque that can be applied to or required to overcome the clamping force of the stabilizer clamp is 21 N-cm.

It will be appreciated that, in some examples, the exemplary delivery system 1000 can include the guide catheter 1100 the stabilizer apparatus 1002, and a prosthetic heart valve delivery apparatus (similar to the delivery apparatus 60 and 300) in place of the docking device delivery apparatus 1200. Specifically, the delivery system 1000 can be used during the third stage of an implant delivery procedure described above with reference to FIG. 3A. In such examples, a cap (such as, the cap 307) of the handle of the prosthetic heart valve delivery apparatus can be coupled to the stabilizer clamp. During operation (for example, steering, navigating, positioning, flexing, etc.) of the prosthetic heart valve delivery apparatus, the handle thereof can be rotated by an operator in each of a clockwise direction and a counterclockwise direction to adjust the implant delivery shaft. Similar to operation of the guide catheter 1100 and the delivery apparatus 1200, it may be desirable for the rotation of the handle in each of the clockwise direction and the counterclockwise direction to require approximately the same amount of force to be applied to the handle. Further, it may be desirable that a position of the handle is maintained when no rotational force is applied to the handle by the operator.

Turning to FIGS. 11-15, exemplary stabilizer clamps that are configured to balance torque resistance for rotation of the handles of a guide catheter apparatus and a delivery apparatus in a first direction and a second opposing direction are shown and described. FIG. 11 illustrates one exemplary stabilizer clamp 1304, in accordance with the present disclosure. Specifically, FIG. 11 shows a jaw portion 1316 of the stabilizer clamp 1304. A base portion of the stabilizer clamp 1304 is not shown, but the base portion of the claim 1304 can have one or more features of the base portions 1008 configured for slidable coupling to a rail discussed above and shown in FIGS. 10A and 10B or can have other features or configurations. For example, the base portion of the stabilizer clamp 1304 can be a stationary base portion mounted to or integral with a stabilizer table. In another example, the base portion of the stabilizer clamp 1304 can include a coupler (for example, a snap-fit coupler, a threaded coupler, etc.) configured to be coupled to a mating partner on a stabilizer table.

As can be seen in FIG. 11, the jaw portion 1316 can include or be formed by a stationary jaw member 1318 and a moveable jaw member 1320. In some examples, the moveable jaw member 1320 can be a lever that rotates over or around a fulcrum 1322 on the stationary jaw member 1318. In other examples, the lever can rotate over or around a fulcrum on the base portion of the stabilizer clamp 1304. Although not shown, the base portion can form one end (for example, a lower end) of stabilizer clamp 1304 and the stationary jaw member 1318 can extend from the base portion. In some examples, the stationary jaw member 1318 can be mounted or attached to the base portion. In some examples, the stationary jaw member 1318 can be integral with the base portion.

The stationary jaw member 1318 and the moveable jaw member 1320 can each include a curved interior wall 1324, 1326 that cooperatively define a mouth portion 1328. In some examples, the mouth portion 1328 can form a generally cylindrical space or void configured to receive a distal cap of a handle of a delivery apparatus or a guide catheter, such as one of the caps 107, 232, 307, 1107, 1232, or other portions of a handle of a delivery apparatus or a guide catheter. The mouth portion 1328 can have an opening 1330 at an opposing end (for example, an upper end) of the stabilizer clamp 1304 relative to the base portion (not shown).

The moveable jaw member 1320 can be configured to be moved or rotated between an open position and a closed position for opening and closing of the mouth 1328. The opening 1330 to the mouth portion can be wider in the open position of the moveable jaw member 1320 to enable insertion (or removal) of a distal cap of a handle of a delivery apparatus or a guide catheter, and can be narrower in the closed position of the moveable jaw member 1320 to enable retaining of the distal cap of the handle therein.

In some examples, a spring member 1332 (which, as discussed below, can be one or more spring members) can be disposed between the stationary jaw member 1318 (or base portion) and the moveable jaw member 1320. The spring member 1332 can be configured to bias the moveable jaw member 1320 toward to the closed position to enable clamping or application of a compressive force on an exterior surface of a distal cap of a handle (or other portion of a handle) disposed within the mouth 1328. Further, the distal cap can be sandwiched or captured between the curved interior walls 1324 and 1326 when the mouth 1328 is in a closed position via the force exerted by the spring member 1332 on the stationary jaw member 1318 and the moveable jaw member 1320.

The moveable jaw member 1320 can include an extension 1334 configured to be depressed by an operator to overcome the biasing force of the spring member 1332 and move the moveable jaw member 1320 into the open position. In some examples, the extension 1334 can include surface features or ribs 1336 configured to facilitate grip on the extension 1334.

As shown in the illustrated example, the spring member 1332 can be a coil member having a first end thereof retained within a first spring seat 1342 within the stationary jaw member 1318 and a second end thereof retained within a second spring seat 1344 within the moveable jaw member 1320. The coil can be comprised a resilient material, such as steel, for example, high-carbon steel, steel alloys, stainless steel, etc., or other resilient materials. In some examples, the spring member can have a different, non-coiled configuration. In some examples, the spring member can be a curved or angled resilient member configured to bow or bend when the extension of the moveable jaw member is depressed and the mouth is moved to the open position. In some examples, the spring member can be a torsion spring member. For example, a torsion spring can be mounted around or at the fulcrum and engage a portion of each of the jaw members.

As discussed above, a spring rate of the spring member 1332 may be sufficient to resist axial rotation of a distal cap and/or handle when no rotational force is exerted upon the handle by an operator (for example, when the handle is initially coupled to the stabilizer clamp or when the operator stops rotating the handle) and/or to resist axial rotation of a distal cap and/or handle from torque built up at the distal end portion of the delivery device while the distal end portion is within the patient's body. In some examples, the spring member 1332 can have a spring rate in a range of 5 lbs./in to 50 lbs./in. In some examples, the spring member 1332 has a minimum spring rate of 30 lbs./in. In some examples, a minimum torque required to overcome the clamping force is in a range of 15 N-cm to 30 N-cm, such as, for example, 18 N-cm to 25 N-cm. In some examples, a minimum torque that can be applied to or required to overcome the clamping force of the stabilizer clamp is 21 N-cm. In some examples, the minimum torque that can be applied to or required to overcome the clamping force of the stabilizer clamp may be representative of a clamping force of the stabilizer clamp.

In some examples, the spring member 1332 can extend between the moveable jaw member 1320 and the stationary jaw member 1318 at an angle due to, for example, the relative positions of the spring seats 1342, 1344. For example, a floor of the first spring seat 1342 can be disposed an angle in a range of 1° to 40°, such as 1° to 25° or 1° to 10°, relative to a longitudinal axis of the stabilizer clamp 1304, and a floor of the second spring seat 1344 can be perpendicular to the longitudinal axis of the stabilizer clamp 1304. In some examples, the spring member 1332 can be parallel with the longitudinal axis of the stabilizer clamp 1304. In such examples, each of the floors of each of the first and second spring seats can be perpendicular to the longitudinal axis of the stabilizer clamp.

In some examples, a greater angle of the spring member can reduce an effective spring rate of the spring member (that is, contribution of a biasing force of the spring to the overall clamping force of the stabilizer clamp), whereas a lesser angle or a parallel position of the spring member can increase the effective spring rate of the spring member. Thus, in some examples, if the spring member is disposed at an angle relative to the longitudinal axis of the stabilizer clamp, a spring with a greater spring rate can be utilized. In some examples, if the spring member is parallel to the longitudinal axis of the stabilizer clamp, a spring with a lower spring rate can be utilized.

Although only a single spring member 1332 is illustrated in FIG. 11, it will be appreciated that one or more additional spring members can be included in the stabilizer clamp. In some examples, the stabilizer clamp can include two (or more) spring members in a side-by-side arrangement or other arrangement, each coupled to or seated within first and second spring seats in or on the stationary jaw member and the moveable jaw member. For example, the stabilizer clamp 1304 can include two identical springs 1332 in a side-by-side configuration. In other words, FIG. 11 can represent an illustration of one side of the stabilizer clamp 1304 including a first spring 1332, and an opposing side of the clamp can have a similar configuration including a second spring 1332.

It will be appreciated that in examples including a greater number of spring members, each individual spring can have a lower spring rate relative to examples including a fewer number of spring members to achieve a sufficient and/or a minimum clamping force. For example, in a stabilizer clamp including two identical springs 1332, each spring can have a spring rate of 30 lbs./in. In a stabilizer claim including one spring 1332, the spring can have a greater spring rate, such as, for example, a spring rate of in a range of 40 to 60 lbs./in.

In some examples, a surface area of the curved interior wall 1326 of the moveable jaw member 1320 can be less than a surface area of the curved interior wall 1324 of the stationary jaw member 1318. In some examples, the curved interior wall 1326 of the moveable jaw member 1320 includes a reduced or recessed lip 1338 at the opening 1330 of the mouth portion 1328. In some examples, when the moveable jaw member 1320 is in the closed position, the reduced or recessed lip 1338 is a greater distance from a vertical axis of the mouth portion than a (non-recessed) lip 1340 of the stationary jaw member 1318.

In such examples, the reduced surface area of the curved interior wall 1326 of the moveable jaw member 1320 at the opening 1330 of the mouth 1328 can reduce torque resistance in a first direction of rotation away from the moveable jaw member (lever) (for example, in a counterclockwise direction in the example of FIG. 11) relative to a curved interior wall that has an identical configuration to the curved interior wall 1324 or a surface area equal to that of the curved interior wall 1324 of the stationary jaw member 1318. For example, a highest point or area of friction may occur or be located at the lip of the curved interior wall of the moveable jaw member when a cap or handle disposed therein is rotated away from the moveable jaw member (for example, in a counterclockwise direction). Accordingly, in some examples, the reduced or recessed lip 1338 can reduce or eliminate the high friction point or area of the curved interior wall 1326 and thereby reduce torque resistance for rotation of the handle attached to or coextensive with the distal cap coupled to the stabilizer clamp 1304 when the handle is rotated away from the moveable jaw member (for example, in a counterclockwise direction in the example of FIG. 11).

In some examples, reduction or elimination of the high friction point or area in of the curved interior wall 1326 of the moveable jaw member 1320 via the recessed lip 1338 can balance torque resistance and/or can bring the torque resistance required to rotate the handle in a direction away from the moveable jaw member (for example, in a counterclockwise direction in the example of FIG. 11) closer to the torque resistance required to rotate the handle in an opposing direction toward the moveable jaw member (for example, in a clockwise direction in the example of FIG. 11).

In some examples, the stabilizer clamp 1304 is configured such that a ratio of torque resistance for rotation of the handle in the first direction relative to torque resistance for rotation of the handle in a second opposing direction is within a range of 0.8 to 1.2. In some examples, the stabilizer clamp 1304 is configured such that a ratio of torque resistance for rotation of the handle in the first direction relative to torque resistance for rotation of the handle in a second opposing direction is within a range of 0.9 to 1.1. In some examples, the stabilizer clamp 1304 is configured such that a ratio of torque resistance for rotation of the handle in the first direction relative to torque resistance for rotation of the handle in a second opposing direction is approximately 1.0.

FIGS. 12 and 13 show exemplary stabilizer clamps 1404 and 1504, in accordance with the present disclosure. Specifically, FIGS. 12 and 13 show upper sections (that is, sections including the mouth portions 1428, 1528) of jaw portions 1416, 1516 of the stabilizer clamps 1404, 1504. Lower sections of the jaw portions 1416, 1516 of the stabilizer clamps 1404, 1504 are not shown, but can have one or more features of the lower section of the stabilizer claim 1304 shown in FIG. 11. For example, the lower sections of the jaw portions 1416, 1516 of the stabilizer clamps 1404, 1504 can include a fulcrum and a spring member seated within spring seats similar to the fulcrum 1322 and the spring member 1332 of the stabilizer clamp 1304. In some examples, the lower section of the jaw portions 1416, 1516 of the stabilizer clamps 1404, 1504 can include additional or alternate features relative to the stabilizer clamp 1304. Further, base portions of the stabilizer clamps 1404, 1504 are not shown, but can have one or more features of the base portions 1008 configured for slidable coupling to a rail discussed above and shown in FIGS. 10A and 10B or can have other features or configurations. For example, the base portion can be a stationary base portion mounted to or integral with a stabilizer table. In another example, the base portion can include a coupler (for example, a snap-fit coupler, a threaded coupler, etc.) configured to be coupled to a mating partner on a stabilizer table.

As can be seen in FIGS. 12 and 13, the jaw portions 1416, 1516 can include or be formed by a stationary jaw member 1418, 1518 and a moveable jaw member 1420, 1520. In some examples, the moveable jaw member 1420, 1520 can be a lever that rotates over or around a fulcrum on the stationary jaw member 1418, 1518. In other examples, the lever can rotate over or around a fulcrum on the base portion of the stabilizer clamp 1404, 1504. Although not shown, the base portion can form one end (for example, a lower end) of stabilizer clamp and the stationary jaw member 1418, 1518 can extend from the base portion. In some examples, the stationary jaw member 1418, 1508 can be mounted or attached to the base portion. In some examples, the stationary jaw member 1418, 1518 can be integral with the base portion.

The stationary jaw member 1418, 1518 can include a curved interior wall 1424, 1524 and the moveable jaw member 1420, 1520 can include a curved interior wall 1426, 1526. The curved interior wall 1424, 1524 and the curved interior wall 1426, 1526 can cooperatively define a mouth portion 1428, 1528 of the clamp 1404, 1504. In some examples, the mouth portion 1428, 1528 can form a generally cylindrical space or void configured to receive a distal cap of a handle of a delivery apparatus or a guide catheter, such as the caps 107, 232, 307, 1107, 1232, or other caps or other portions of a handle of a delivery apparatus or a guide catheter. The mouth portion 1428, 1528 can have an opening 1430, 1530 at an opposing end (for example, an upper end) of the stabilizer clamp 1404, 1504 relative to the base portion (not shown).

The moveable jaw member 1420, 1520 can be configured to be moved or rotated between an open position and a closed position for opening and closing of the mouth 1428, 1528. The opening 1430, 1530 to the mouth portion 1428, 1528 can be wider in the open position of the moveable jaw member 1420, 1520 to enable insertion (or removal) of a distal cap of a handle, and can be narrower in the closed position of the moveable jaw member 1420, 1520 to enable retaining of the distal cap of the handle therein.

As discussed above with reference to the stabilizer clamp 1304 of FIG. 11, in some examples a spring member can bias the moveable jaw member toward to the closed position to enable clamping or application of a compressive force on an exterior surface of a distal cap or handle disposed within the mouth 1428, 1528. Further, the moveable jaw member 1420, 1520 can include an extension 1434, 1534 configured to be depressed by an operator to overcome the biasing force of the spring member and move the moveable jaw member 1420, 1520 into the open position. Furthermore, the spring member of the stabilizer clamp 1404, 1504 can have one or more of the features discussed above with reference to the spring member 1332 or additional or alternate features.

In some examples, such as in the exemplary stabilizer clamp 1404 illustrated in FIG. 12, a surface area of the curved interior wall 1426 of the moveable jaw member 1420 can be less than a surface area of the curved interior wall 1424 of the stationary jaw member 1418. In some examples, the curved interior wall 1426 of the moveable jaw member 1420 can include a reduced or recessed lip 1438 at the opening 1430 of the mouth portion 1428. In some examples, when the moveable jaw member 1420 is in the closed position, the reduced or recessed lip 1438 is a greater distance from a vertical axis of the mouth portion than a (non-recessed) lip 1440 of the stationary jaw member 1418.

In such examples, the reduced surface area of the curved interior wall 1426 of the moveable jaw member 1420 at the opening 1430 of the mouth 1428 can reduce torque resistance in a first direction of rotation away from the moveable jaw member (lever) (for example, in a counterclockwise direction in the example of FIG. 12) relative to a curved interior wall that has an identical configuration to the curved interior wall 1424 or a surface area equal to that of the curved interior wall 1424 of the stationary jaw member 1418. For example, a highest point or area of friction may occur or be located at the lip of the curved interior wall of the moveable jaw member when a cap disposed therein is rotated away from the moveable jaw member (for example, in a counterclockwise direction). Accordingly, in some examples, the reduced or recessed lip 1438 can reduce or eliminate the high friction point or area of the curved interior wall 1426 and thereby reduce torque resistance for rotation of the handle attached to or coextensive with the cap when the handle is rotated away from the moveable jaw member (for example, in a counterclockwise direction in the example of FIG. 12).

Additionally, as schematically illustrated in FIG. 12, the curved interior wall 1426 can include a textured surface or a coated surface 1446. In some examples, the textured or coated surface 1446 is a graduated or a gradient surface including a lower friction area at a first end region 1448 of the curved interior wall 1426 (for example, at an end region of the curved interior wall 1426 proximate the lip 1438) and a higher friction area at a second end region 1450 of the curved interior wall 1426 (for example, at an opposing end region of the curved interior wall 1426 relative to the lip 1438). In some examples, the lower friction area can comprise a smoother surface and/or a lower friction material coating and the higher friction area can comprise a rougher surface and/or a higher friction material coating. For example, the surface 1446 can include a plastic substrate having silicone or rubber protrusions (for example, bumps or beads) disposed thereon. The higher friction area at the second end region 1450 of the curved interior wall 1426 can include a higher density of the protrusions relative to the lower friction area at the first end region 1448.

In such examples, reduction or elimination of the high friction point or area in of the curved interior wall 1426 of the moveable jaw member 1420 via the recessed lip 1438 in combination with the textured surface gradient 1446 on the curved interior wall can balance torque resistance and/or can bring the torque resistance required to rotate the handle in a direction away from the moveable jaw member (for example, in a counterclockwise direction in the example of FIG. 12) closer to the torque resistance required to rotate the handle in an opposing direction toward the moveable jaw member (for example, in a clockwise direction in the example of FIG. 12).

In some examples, the stabilizer clamp 1404 is configured such that a ratio of torque resistance for rotation of the handle in the first direction relative to torque resistance for rotation of the handle in a second opposing direction is within a range of 0.8 to 1.2. In some examples, the stabilizer clamp 1404 is configured such that a ratio of torque resistance for rotation of the handle in the first direction relative to torque resistance for rotation of the handle in a second opposing direction is within a range of 0.9 to 1.1. In some examples, the stabilizer clamp 1404 is configured such that a ratio of torque resistance for rotation of the handle in the first direction relative to torque resistance for rotation of the handle in a second opposing direction is approximately 1.0.

In some examples, a graduated or gradient textured surface on the curved interior wall of the moveable jaw member is sufficient for balancing torque resistance, and the curved interior wall of the moveable jaw member can include a non-recessed lip or can have a configuration similar to the curved interior wall of the stationary jaw member. For example, as shown in FIG. 13, the curved interior wall 1526 of the moveable jaw member 1520 can have an approximately equal or similar surface area to the curved interior wall 1524 of the stationary jaw member 1518. In one specific example, a lip 1538 of the curved interior wall 1526 can have a similar configuration to and/or be non-recessed relative to a lip 1540 of the curved interior wall 1524.

As schematically shown in FIG. 13, the curved interior wall 1526 can include a textured surface or a coated surface 1546. In some examples, the textured or coated surface 1546 is a graduated or a gradient surface including a lower friction area at a first end region 1548 of the curved interior wall 1526 (for example, at an end region of the curved interior wall 1526 proximate the lip 1538) and a higher friction area at a second end region 1550 of the curved interior wall 1526 (for example, at an opposing end region of the curved interior wall 1526 relative to the lip 1538). In some examples, the lower friction area can comprise a smoother surface and/or a lower friction material coating and the higher friction area can comprise a rougher surface and/or a higher friction material coating. For example, the textured surface 1546 can include a plastic substrate having silicone or rubber protrusions (for example, bumps or beads) disposed thereon. The higher friction area at the second end region 1550 of the curved interior wall 1526 can include a higher density of the protrusions relative to the lower friction area at the first end region 1548.

In such examples, the textured surface gradient 1546 on the curved interior wall 1526 can balance torque resistance and/or can bring the torque resistance required to rotate the handle in a direction away from the moveable jaw member (for example, in a counterclockwise direction in the example of FIG. 13) closer to the torque resistance required to rotate the handle in an opposing direction toward the moveable jaw member (for example, in a clockwise direction in the example of FIG. 13).

In some examples, the stabilizer clamp 1504 is configured such that a ratio of torque resistance for rotation of the handle in the first direction relative to torque resistance for rotation of the handle in a second opposing direction is within a range of 0.8 to 1.2. In some examples, the stabilizer clamp 1504 is configured such that a ratio of torque resistance for rotation of the handle in the first direction relative to torque resistance for rotation of the handle in a second opposing direction is within a range of 0.9 to 1.1. In some examples, the stabilizer clamp 1504 is configured such that a ratio of torque resistance for rotation of the handle in the first direction relative to torque resistance for rotation of the handle in a second opposing direction is approximately 1.0.

In some examples, the curved interior wall 1424 (FIG. 12) and/or the curved interior wall 1524 (FIG. 13) can include a coating or textured surface. In some examples, the curved interior walls 1424, 1524 can include a surface comprising a gradient extending from a higher friction area to a lower friction area. In some examples, the curved interior walls 1424, 1524 can include a surface comprising continuous surface having a desired friction coefficient (for example, a higher friction coefficient surface or a lower friction coefficient surface). In some examples, the coating or textured surface on the curved interior walls 1424, 1524 can enable balance of a torque resistance for rotating a handle coupled to the clamp in a first direction relative to torque resistance for rotating the handle in a second direction. For example, the coating or textured surface on the curved interior walls 1424, 1524 in combination with the coating or textured surface on the curved interior walls 1426, 1526 can enable a desired or specified ratio of torque resistance for rotation of the handle in the first direction relative to torque resistance for rotation of the handle in a second opposing direction (such as, for example, a ratio within and/or corresponding to the exemplary ranges discussed above).

In some examples, where the curved interior wall 1424, 1524 on the stationary jaw member 1418, 1518 includes a coating or textured surface, the opposing curved interior wall 1426, 1526 on the moveable jaw member 1420, 1520 can be a smooth or low friction surface. In such examples, the stationary jaw member 1418, 1518 can provide all or a majority of the friction needed to retain a position of a distal cap (or other portion) of a handle of a delivery apparatus or a guide catheter within the mouth portion 1428, 1528, while little friction is applied to the distal cap by the movable jaw member 1420, 1520. In such examples, opening and closing of the moveable jaw member 1420, 1520 can be reduced and/or minimized during application torque to the handle, and can thereby equalize and/or balance the torques for rotation of the handle in counterclockwise and clockwise directions. For example, a ratio of torque resistance for rotation of the handle in the first direction relative to torque resistance for rotation of the handle in a second opposing direction can be within a range of 0.8 to 1.2, such as a range of 0.9 to 1.1, or the ratio can be approximately 1.0.

FIG. 14 illustrates another exemplary stabilizer clamp 1604, in accordance with the present disclosure. Specifically, FIG. 14 shows a jaw portion 1616 of the stabilizer clamp 1604. A base portion of the stabilizer clamp 1604 is not shown, but can have one or more features of the base portions 1008 configured for slidable coupling to a rail discussed above and shown in FIGS. 10A and 10B or can have other features or configurations. For example, the base portion of the stabilizer clamp 1604 can be a stationary base portion mounted to or integral with a stabilizer table. In another example, the base portion can include a coupler (for example, a snap-fit coupler, a threaded coupler, etc.) configured to be coupled to a mating partner on a stabilizer table.

As can be seen in FIG. 14, the jaw portion 1616 can include or be formed by a stationary jaw member 1618 and a moveable jaw member 1620. In some examples, the moveable jaw member 1620 can be a lever that rotates over or around a fulcrum 1622 on the stationary jaw member 1618. In other examples, the lever can rotate over or around a fulcrum on the base portion of the stabilizer clamp. Although not shown, the base portion can form one end (for example, a lower end) of stabilizer clamp 1604 and the stationary jaw member 1618 can extend from the base portion. In some examples, the stationary jaw member 1618 can be mounted or attached to the base portion. In some examples, the stationary jaw member 1618 can be integral with the base portion.

The stationary jaw member 1618 and the moveable jaw member 1620 can each include a curved interior wall 1624, 1626 that cooperatively define a mouth portion 1628. In some examples, the mouth portion 1628 can form a generally cylindrical space or void configured to receive a distal cap of a handle of a delivery apparatus or a guide catheter, such as the caps 107, 232, 307, 1107, 1232, or other portions of a handle of a delivery apparatus or a guide catheter. The mouth portion 1628 can have an opening 1630 at an opposing end (for example, an upper end) of the stabilizer clamp 1604 relative to the base portion (not shown).

The moveable jaw member 1620 can be configured to be moved or rotated between an open position and a closed position for opening and closing of the mouth 1628. The opening 1630 to the mouth portion 1628 can be wider in the open position of the moveable jaw member 1620 to enable insertion (or removal) of a distal cap of a handle, and can be narrower in the closed position of the moveable jaw member 1620 to enable retaining of the distal cap of the handle therein.

In some examples, a spring member 1632 can be disposed between the stationary jaw member 1618 (or base portion) and the moveable jaw member 1620. The spring member 1632 can be configured to bias the moveable jaw member 1620 toward to the closed position to enable clamping or application of a compressive force on an exterior surface of a distal cap (or other portion of a handle) disposed within the mouth 1628. Further, a distal cap can be sandwiched or captured between the curved interior walls 1624 and 1626 when the mouth 1628 is in the closed position via the force exerted by the spring member 1632 on the stationary jaw member 1618 and the moveable jaw member 1620. As shown in the illustrated example, the spring member 1632 can be a coil member having a first end thereof retained within a spring seat 1642 within the stationary jaw member 1618 and a second end thereof retained within a spring seat 1644 withing the moveable jaw member 1320. In some examples, the spring member 1632 has one or more of the features and/or variations discussed above with reference to the spring member 1332 of FIG. 11. In some examples, the spring member 1632 can be one or more spring members, such as, for example, two spring member 1632 in a side-by-side arrangement. In some examples, the spring member 1632 includes additional or alternate features.

The moveable jaw member 1620 can include an extension 1634 configured to be depressed by an operator to overcome the biasing force of the spring member 1632 and move the moveable jaw member 1620 into the open position. In some examples, the extension 1634 can include surface features or ribs 1636 configured to facilitate grip on the extension 1634.

Different from the stabilizer clamp 1304, the stabilizer clamp 1604 can further include a locking mechanism or lock 1652 configured to selectively retain or lock a position of the moveable jaw member 1620 relative to the stationary jaw member 1618. In some examples, the locking mechanism 1652 comprises an actuator or control 1654 (for example, a button, a switch, a slider, etc.) disposed on the extension 1634 that can be actuated by a user to engage and/or disengage a shaft 1656 of the locking mechanism 1652 from the stationary jaw member 1618 or the base portion (not shown). When the shaft 1656 of the locking mechanism is engaged (as depicted in FIG. 14), movement of the moveable jaw member 1620 is limited relative to the stationary jaw member 1618 and the mouth portion 1628 can be retained in the closed position. When the shaft 1656 of the locking mechanism is disengaged (not shown), the moveable jaw member 1620 is rotatable or moveable relative to the stationary jaw member 1618 and the mouth portion 1628 can be moved to the open position.

In some examples, a surface area of the curved interior wall 1626 of the moveable jaw member 1620 can be less than a surface area of the curved interior wall 1624 of the stationary jaw member 1618. In some examples, the curved interior wall 1626 of the moveable jaw member 1620 includes a reduced or recessed lip 1638 at the opening 1630 of the mouth portion 1628. In some examples, when the moveable jaw member 1420 is in the closed position, the reduced or recessed lip 1638 is a greater distance from a vertical axis of the mouth portion than a (non-recessed) lip 1640 of the stationary jaw member 1618.

In such examples, the reduced surface area of the curved interior wall 1626 of the moveable jaw member 1620 at the opening 1630 of the mouth 1628 can reduced torque resistance in a first direction of rotation away from the moveable jaw member (lever) (for example, a counterclockwise direction in the example of FIG. 14) relative to a curved interior wall that has an identical configuration to the curved interior wall 1624 or a surface area equal to that of the curved interior wall 1624 of the stationary jaw member 1618. For example, a highest point or area of friction may occur or be located at the lip of the curved interior wall of the moveable jaw member when a cap disposed therein is rotated away from the moveable jaw member (for example, in a counterclockwise direction). Accordingly, in some examples, the reduced or recessed lip 1638 can reduce or eliminate the high friction point or area of the curved interior wall 1626 and thereby reduce torque resistance for rotation of the handle attached to or coextensive with a distal cap when the handle is rotated away from the moveable jaw member 1620 (for example, in a counterclockwise direction in the example of FIG. 14).

Though not illustrated, in some examples, the curved interior wall 1626 can additionally include a textured surface or coating, or textured surface or coating gradient similar to the textured surface gradient 1446 discussed above with reference to FIG. 14. In some examples including a textured surface gradient, a lip of the curved interior wall 1626 can be non-recessed or can have a similar configuration to the lip 1640 of the curved interior wall of the stationary jaw member 1618 (similar to the stabilizer clamp 1504 illustrated in FIG. 13). In some examples, the curved interior wall 1624 can include a textured or coated surface that can be a gradient from a higher friction area to a lower friction area, or can be a continuous surface with a specified friction coefficient (as discussed above).

In some examples, reduction or elimination of the high friction point or area in of the curved interior wall 1626 of the moveable jaw member 1620 via inclusion of the recessed lip 1638 and/or inclusion of a textured surface gradient on the curved interior wall 1626 can balance torque resistance and/or can bring the torque resistance required to rotate the handle in a direction away from the moveable jaw member (for example, in a counterclockwise direction in the example of FIG. 14) closer to the torque resistance required to rotate the handle in an opposing direction toward the moveable jaw member (for example, in a clockwise direction in the example of FIG. 14).

Additionally, in examples including a recessed lip and/or a textured surface gradient in the curved interior wall of the moveable member, when the shaft 1656 of the locking mechanism 1652 is engaged, the locking mechanism 1652 can further balance torque resistance and/or can bring the torque resistance required to rotate the handle in a direction away from the moveable jaw member (for example, in a counterclockwise direction in the example of FIG. 14) closer to the torque resistance required to rotate the handle in an opposing direction toward the moveable jaw member (for example, in a clockwise direction in the example of FIG. 14) by resisting or limiting movement of the moveable member 1620.

Alternatively, in examples excluding a recessed lip and/or a textured surface gradient in the curved interior wall of the moveable member, when the shaft 1656 of the locking mechanism 1652 is engaged, the locking mechanism 1652 can balance torque resistance and/or can bring the torque resistance required to rotate the handle in a direction away from the moveable jaw member (for example, in a counterclockwise direction in the example of FIG. 14) closer to the torque resistance required to rotate the handle in an opposing direction toward the moveable jaw member (for example, in a clockwise direction in the example of FIG. 14) by resisting or limiting movement of the moveable member 1620.

In some examples, the stabilizer clamp 1604 is configured such that a ratio of torque resistance for rotation of the handle in the first direction relative to torque resistance for rotation of the handle in a second opposing direction is within a range of 0.8 to 1.2. In some examples, the stabilizer clamp 1604 is configured such that a ratio of torque resistance for rotation of the handle in the first direction relative to torque resistance for rotation of the handle in a second opposing direction is within a range of 0.9 to 1.1. In some examples, the stabilizer clamp 1604 is configured such that a ratio of torque resistance for rotation of the handle in the first direction relative to torque resistance for rotation of the handle in a second opposing direction is approximately 1.0.

It will be appreciated that the illustrated locking mechanism 1652 is merely exemplary, and the stabilizer clamp 1604 can include a locking mechanism having a different configuration. For example, a locking mechanism can include a post or threaded member that is insertable through a portion of the moveable jaw member 1620 and the stationary jaw member 1618 to retain a position or limit movement of the moveable member jaw 1620 relative to the stationary jaw member 1618.

FIG. 15 shows another exemplary stabilizer clamp 1704, in accordance with the present disclosure. Specifically, FIG. 15 shows a jaw portion 1716 of the stabilizer clamp 1704. A base portion of the stabilizer clamp 1704 is not shown, but can have one or more features of the base portions 1008 configured for slidable coupling to a rail discussed above and shown in FIGS. 10A and 10B or can have other features or configurations. For example, the base portion of the stabilizer clamp 1704 can be a stationary base portion mounted to or integral with a stabilizer table. In another example, the base portion can include a coupler (for example, a snap-fit coupler, a threaded coupler, etc.) configured to be coupled to a mating partner on a stabilizer table.

Different from the stabilizer clamps 1304, 1404, 1504, 1604, the stabilizer clamp 1704 can be a dual-lever stabilizer clamp. For example, as illustrated in FIG. 15, the jaw portion 1716 can include or be formed by a first moveable jaw member 1720a and a second moveable jaw member 1720b. In some examples, each of the moveable jaw members 1720a, 1720b can be a lever that rotates over or around a fulcrum 1722 on a stationary portion 1758 of the stabilizer clamp 1704 that is coextensive with or attached to the base portion. In other examples, the levers can rotate over or around a fulcrum on the base portion of the stabilizer clamp 1704.

The moveable jaw members 1720a, 1720b can each include a curved interior wall 1726a, 1726b that cooperatively define a mouth portion 1728. In some examples, the mouth portion 1728 can form a generally cylindrical space or void configured to receive a distal cap of a handle of a delivery apparatus or a guide catheter, such as the caps 107, 232, 307, 1107, 1232, or other portions of a handle of a delivery apparatus or a guide catheter. The mouth portion 1728 can have an opening 1730 at an opposing end (for example, an upper end) of the stabilizer clamp 1704 relative to the base portion (not shown).

The moveable jaw members 1720a, 1720b can be configured to be moved or rotated between an open position and a closed position for opening and closing of the mouth 1728. The opening 1730 to the mouth portion 1728 can be wider in the open position of the moveable jaw members 1720a, 1720b to enable insertion (or removal) of a distal cap of a handle, and can be narrower in the closed position of the moveable jaw members 1720a, 1720b to enable retaining of the distal cap of the handle therein.

In some examples, spring members 1732a, 1732b can be disposed between the stationary portion 1758 (or base portion) and the moveable jaw members 1720a, 1720b. The spring members 1732a, 1732b can be configured to bias the moveable jaw members 1720a, 1720b toward to the closed position to enable clamping or application of a compressive force on an exterior surface of a distal cap (or other portion of a handle) disposed within the mouth 1728. Further, a distal cap can be sandwiched or captured between the curved interior walls 1726a and 1726b when the mouth 1728 is in the closed position via the force exerted by the spring members 1732a, 1732b on the stationary portion 1758 and the moveable jaw members 1720a, 1720b. As shown in the illustrated example, the spring members 1732a, 1732b can be coil members having a first end thereof retained within a spring seat 1742a, 1742b within the stationary portion 1758 and a second end thereof retained within a spring seat 1744a, 1744b within the moveable jaw members 1720a, 1720b.

In some examples, the spring members 1732a, 1732b have one or more of the features and/or variations discussed above with reference to the spring member 1332 of FIG. 11. In some examples, each of the spring members 1732a, 1732b can be one or more spring members, such as, for example, two spring member 1732a in a side-by-side arrangement and two spring members 1732b in a side-by-side arrangement. In some examples, the spring members 1732a, 1732b include additional or alternate features. In some examples, the spring members 1732a, 1732b have an identical configuration or orientation. In some examples, the spring members 1732a, 1732b each have a different configuration or orientation.

The moveable jaw members 1720a, 1720b can include extensions 1734a, 1734b configured to be depressed by an operator to overcome the biasing force of the spring members 1732a, 1732b and move the moveable jaw members 1720a, 1720b into the open position.

In some examples, a configuration of each of the curved interior walls 1726a, 1726b can be similar or identical. For example, a surface area and shape of a lip 1738a, 1738b at the opening 1730 of the mouth portion 1728 can be identical or similar for each of the curved interior walls 1726a, 1726b. In some examples, the curved interior walls 1726a, 1726b can have different configurations.

In some examples, the curved interior walls 1726a, 1726b can additionally include a textured surface or coating or textured or coated surface gradient or can be a continuous surface with a specified friction coefficient similar to the examples discussed above with reference to FIGS. 14 and 15.

In some examples, the stabilizer clamp 1704 can further include a coupler 1760 disposed between the moveable jaw members 1720a, 1720b. In some examples, the coupler can be configured to link the moveable jaw members 1720a, 1720b such that opening and/or rotation of one of the moveable jaw members 1720a, 1720b causes or results in opening or rotation of the other of the moveable jaw members 1720a, 1720b. For example, depression of the extension 1734a causes rotation of the moveable jaw member 1720a away from a longitudinal axis of the stabilizer clamp 1704, which, via the coupler 1760, results in rotation of the moveable jaw member 1720b away from the longitudinal axis of the stabilizer clamp 1704. In another example, depression of the extension 1734b causes rotation of the moveable jaw member 1720b away from the longitudinal axis of the stabilizer clamp 1704, which, via the coupler 1760, results in rotation of the moveable jaw member 1720a away from the longitudinal axis of the stabilizer clamp 1704.

In some examples, the dual levers (moveable jaw members 1720a, 1720b) having the coupler 1760 disposed therebetween can balance torque resistance for rotation of the handle in a first direction (for example, in a counterclockwise direction in the example of FIG. 15) and rotation of the handle in a second opposing direction (for example, in a clockwise direction in the example of FIG. 15). For example, in the illustrated example, rotation of a distal cap of a handle disposed within the mouth portion 1728 in a counterclockwise direction can cause (at least some) movement or rotation of the moveable jaw member 1720a away from a longitudinal axis of the stabilizer clamp 1704, which, via the coupler 1760, results in rotation of the moveable jaw member 1720b away from the longitudinal axis of the stabilizer clamp 1704. In another example, rotation of a distal cap of a handle disposed within the mouth portion 1728 in a clockwise direction can cause (at least some) movement or rotation of the moveable jaw member 1720b away from the longitudinal axis of the stabilizer clamp 1704, which, via the coupler 1760, results in rotation of the moveable jaw member 1720a away from the longitudinal axis of the stabilizer clamp 1704. In some examples, the coupler 1760 can be excluded from the stabilizer clamp 1704. For example, the moveable jaw members 1720a, 1720b can be independently moveable, and a similar torque can be required for rotation of a handle coupled thereto in a first direction relative to rotation of the handle in the second direction. In such examples, rotating a handle in a counterclockwise direction can cause the moveable jaw member 1720a to move away from the central axis while causing the moveable jaw member 1720b to pull toward the central axis, and rotating the handle in a clockwise direction can cause the moveable jaw member 1720b to move away from the central axis while causing the moveable jaw member 1720a to pull toward the central axis. In some examples, the stabilizer clamp 1704 can further include a stop structure (for example, a stop structure on or near the fulcrum 1722) for each of the moveable jaw members 1720a, 1720b such that when one of the jaw members is opened to insert or remove a cap or other portion of a handle, the opposing jaw member can remain in place and not rotate past the central axis of the stabilizer clamp.

In some examples, the stabilizer clamp 1704 is configured such that a ratio of torque resistance for rotation of the handle in the first direction relative to torque resistance for rotation of the handle in a second opposing direction is within a range of 0.8 to 1.2. In some examples, the stabilizer clamp 1704 is configured such that a ratio of torque resistance for rotation of the handle in the first direction relative to torque resistance for rotation of the handle in a second opposing direction is within a range of 0.9 to 1.1. In some examples, the stabilizer clamp 1704 is configured such that a ratio of torque resistance for rotation of the handle in the first direction relative to torque resistance for rotation of the handle in a second opposing direction is approximately 1.0.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus system comprising: a delivery apparatus comprising: a handle; and a shaft extending distally from the handle, the shaft configured for delivery of an implantable device; and a stabilizer clamp comprising a base portion and a jaw portion extending from the base portion; wherein the jaw portion comprises: a first jaw member comprising a first curved interior wall; and a second jaw member comprising a second curved interior wall, wherein the first curved interior wall and the second curved interior wall form a mouth configured to receive a portion of the handle, and wherein at least one of the first jaw member or the second jaw member is a lever rotatable around a fulcrum for opening and closing of the mouth; and at least one spring member configured to bias the lever towards a closed position of the mouth; and wherein, when the portion of the handle is received within the mouth, a ratio of torque resistance for rotation of the handle in a first direction relative to torque resistance for rotation of the handle in a second opposing direction is within a range of 0.8 to 1.2.

Example 2. The delivery apparatus system of any example disclosed herein, particularly example 1, wherein the first jaw member is a first lever rotatably attached to the base and the second jaw member is a stationary member which is attached to and non-moveable relative to the base.

Example 3. The delivery apparatus system of any example disclosed herein, particularly examples 1 or 2, wherein the first curved interior wall has a decreased surface area relative to the second curved interior wall.

Example 4. The delivery apparatus system of any example disclosed herein, particularly examples 1-3, wherein the first curved interior wall comprises a first upper lip defining a first side of an opening of the mouth and the second curved interior wall comprises a second upper lip defining a second side of the opening of the mouth.

Example 5. The delivery apparatus system of any example disclosed herein, particularly example 4, wherein the first upper lip is recessed relative to the second upper lip.

Example 6. The delivery apparatus system of any example disclosed herein, particularly examples 4 or 5, wherein the first curved interior wall further comprises a first lower lip opposing the first upper lip, and wherein the first curved interior wall has a surface gradient comprising a higher friction surface in a region of the first lower lip and a lower friction surface in a region the first upper lip.

Example 7. The delivery apparatus system of any example disclosed herein, particularly examples 1-6, wherein the stabilizer clamp further comprises a lock configured to retain a position the lever such that the mouth is locked in the closed position.

Example 8. The delivery apparatus system of any example disclosed herein, particularly example 7, wherein the lever further comprises an extension configured to be depressed to rotate the lever to an open position of the mouth, and wherein the lock comprises an actuator on the extension for releasing the lock.

Example 9. The delivery apparatus system of any example disclosed herein, particularly example 1, wherein the first jaw member is a first lever and the second jaw member is a second lever, each of the first lever and the second lever rotatably attached to the base portion, and wherein the at least one spring comprises a first spring configured to bias the first lever towards the closed position of the mouth and a second spring member configured to bias the second lever towards the closed position of the mouth.

Example 10. The delivery apparatus system of any example disclosed herein, particularly example 9, wherein the first lever and the second lever are coupled to each other such that application of a rotational force towards the open position of the mouth on one of the first lever or the second lever to results in opening of the other of the first lever or the second lever.

Example 11. The delivery apparatus system of any example disclosed herein, particularly examples 1-10, wherein, when the portion of the handle is received within the mouth, a minimum torque required for rotation of the handle in one or more of the first direction or the second opposing direction is in a range of 15 N-cm to 30 N-cm.

Example 12. The delivery apparatus system of any example disclosed herein, particularly examples 1-11, wherein the base portion is configured to be slidably coupled to a stabilizer rail.

Example 13. The delivery apparatus system of any example disclosed herein, particularly example 12, wherein the base portion comprise a locking mechanism configured to fix a position of the base portion of the stabilizer clamp on the stabilizer rail.

Example 14. A stabilizer clamp configured for use with an implant delivery apparatus, the stabilizer clamp comprising: a base portion; and a jaw portion extending from the base portion, the jaw portion comprising: a first jaw member comprising a first curved interior wall; and a second jaw member comprising a second curved interior wall, wherein the first curved interior wall and the second curved interior wall form a mouth configured to receive a portion of a handle of the implant delivery apparatus, and wherein at least one of the first jaw member or the second jaw member is a lever rotatable around a fulcrum for opening and closing of the mouth; and at least one spring member configured to bias the lever towards a closed position of the mouth; and wherein the stabilizer clamp is configured such that, when the portion of the handle is received within the mouth, a ratio of torque resistance for rotation of the handle in a first direction relative to torque resistance for rotation of the handle in a second opposing direction is within a range of 0.8 to 1.2.

Example 15. The stabilizer clamp of any example disclosed herein, particularly example 14, wherein the first jaw member is a first lever rotatably attached to the base and the second jaw member is a stationary member which is attached to and non-moveable relative to the base.

Example 16. The stabilizer clamp of any example disclosed herein, particularly example 15, wherein the stabilizer clamp further comprises a lock configured to retain a position the first lever such that the mouth is locked in the closed position.

Example 17. The stabilizer clamp of any example disclosed herein, particularly examples 14-16, wherein the first curved interior wall comprises a first upper lip defining a first side of an opening of the mouth and the second curved interior wall comprises a second upper lip defining a second side of the opening of the mouth.

Example 18. The stabilizer clamp of any example disclosed herein, particularly example 17, wherein the first upper lip is recessed relative to the second upper lip, and the first curved interior wall has a decreased surface area relative to the second curved interior wall.

Example 19. The stabilizer clamp of any example disclosed herein, particularly examples 17 or 18, wherein the first curved interior wall further comprises a first lower lip opposing the first upper lip, and wherein the first curved interior wall has a textured surface gradient comprising a high friction surface in a region of the first lower lip and a low friction surface in a region the first upper lip.

Example 20. The stabilizer clamp of any example disclosed herein, particularly example 14, wherein the first jaw member is a first lever and the second jaw member is a second lever, each of the first lever and the second lever rotatably attached to the base portion, and wherein the at least one spring comprises a first spring configured to bias the first lever towards the closed position of the mouth and a second spring member configured to bias the second lever towards the closed position of the mouth.

Example 21. The stabilizer clamp of any example disclosed herein, particularly example 20, wherein the first lever and the second lever are coupled to each other such that application of a rotational force towards the open position of the mouth on one of the first lever or the second lever to results in opening of the other of first lever or the second lever.

Example 22. The stabilizer clamp of any example disclosed herein, particularly examples 14-21, wherein the stabilizer clamp is configured such that, when the portion of the handle is received within the mouth, a minimum torque applied to overcome a clamping force of the stabilizer clamp is at least 21 N-cm.

Example 23. A method of operating a delivery apparatus system, the method comprising: inserting a portion of a first handle of a first delivery apparatus into a mouth of a first stabilizer clamp, the first stabilizer clamp comprising a base portion attached to a stabilizer rail or table and a first jaw member and a second jaw member extending from the base portion, the mouth defined by a first curved interior wall of the first jaw member and a second curved interior wall of a second jaw member, wherein at least one of the first jaw member or the second jaw member is a lever rotatable around a fulcrum for opening and closing of the mouth, and wherein the first stabilizer clamp further comprises at least one spring member configured to bias the lever towards a closed position of the mouth; rotating the first handle in a first direction to transmit torque to a first shaft coupled to the first handle; and rotating the first handle in a second opposing direction to transit torque to the first shaft coupled to the first handle; wherein the first stabilizer clamp is configured such that, when the portion of the first handle is received within the mouth thereof, a ratio of torque resistance for rotation of the first handle in the first direction relative to torque resistance for rotation of the first handle in the second opposing direction is within a range of 0.8 to 1.2.

Example 24. The method of any example disclosed herein, particularly example 23, further comprising actuating a rotatable knob on the first handle, wherein a clamping force of the stabilizer clamp resists movement of the first handle during the actuating of the rotatable knob.

Example 25. The method of any example disclosed herein, particularly example 24, wherein the stabilizer clamp is configured such that, when the portion of the handle is received within the mouth, a minimum torque for overcoming a clamping force of the stabilizer clamp is at least 21 N-cm.

Example 26. The method of any example disclosed herein, particularly examples 23-25, wherein the first jaw member is a first lever rotatably attached to the base and the second jaw member is a stationary member which is attached to and non-moveable relative to the base.

Example 27. The method of any example disclosed herein, particularly example 26, wherein the stabilizer clamp further comprises a lock configured to retain a position the first lever such that the mouth is locked in the closed position.

Example 28. The method of any example disclosed herein, particularly examples 26 or 27, wherein the first curved interior wall comprises a first upper lip defining a first side of an opening of the mouth and the second curved interior wall comprises a second upper lip defining a second side of the opening of the mouth.

Example 29. The method of any example disclosed herein, particularly example 28, wherein the first upper lip is recessed relative to the second upper lip, and the first curved interior wall has a decreased surface area relative to the second curved interior wall.

Example 30. The method of any example disclosed herein, particularly examples 28 or 29, wherein the first curved interior wall further comprises a first lower lip opposing the first upper lip, and wherein the first curved interior wall has a textured surface gradient comprising a higher friction surface in a region of the first lower lip and a lower friction surface in a region the first upper lip.

Example 31. The method of any example disclosed herein, particularly examples 23-25, wherein the first jaw member is a first lever and the second jaw member is a second lever, each of the first lever and the second lever rotatably attached to the base portion, and wherein the at least one spring comprises a first spring member configured to bias the first lever towards the closed position of the mouth and a second spring member configured to bias the second lever towards the closed position of the mouth.

Example 32. The method of any example disclosed herein, particularly example 31, wherein the first lever and the second lever are coupled to each other such that application of a rotational force towards the open position of the mouth on one of the first lever or the second lever to results in opening of the other of first lever or the second lever.

Example 33. The method of any example disclosed herein, particularly examples 23, further comprising: inserting a delivery shaft of a second delivery apparatus through a lumen of the first handle and the first shaft, wherein the first shaft is a guide catheter, and wherein the delivery shaft includes an implantable device coupled at a distal end region thereof; and inserting a portion of a second handle of the second delivery apparatus into a mouth of a second stabilizer clamp, the second stabilizer clamp comprising a base portion attached to the stabilizer rail or table and a first jaw member and a second jaw member extending from the base portion, the mouth defined by a first curved interior wall of the first jaw member and a second curved interior wall of a second jaw member, wherein at least one of the first jaw member or the second jaw member is a lever rotatable around a fulcrum for opening and closing of the mouth, and wherein the second stabilizer clamp further comprises at least one spring member configured to bias the lever towards a closed position of the mouth; rotating the second handle in a first direction to transmit torque to the delivery shaft coupled to the second handle; and rotating the second handle in a second opposing direction to transit torque to the delivery shaft coupled to the second handle; wherein the second stabilizer clamp is configured such that, when the portion of the second handle is received within the mouth thereof, a ratio of torque resistance for rotation of the second handle in the first direction relative to torque resistance for rotation of the second handle in the second opposing direction is within a range of 0.8 to 1.

The features described herein with regard to any example can be combined with other features described in any one or more of the other examples, unless otherwise stated. For example, any one or more of the features of one stabilizer clamp can be combined with any one or more features of another stabilizer clamp. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus system comprising:
a delivery apparatus comprising:
a handle; and
a shaft extending distally from the handle, the shaft configured for delivery of an implantable device; and
a stabilizer clamp comprising a base portion and a jaw portion extending from the base portion;
wherein the jaw portion comprises:
a first jaw member comprising a first curved interior wall; and
a second jaw member comprising a second curved interior wall, wherein the first curved interior wall and the second curved interior wall form a mouth configured to receive a portion of the handle, and wherein at least one of the first jaw member or the second jaw member is a lever rotatable around a fulcrum for opening and closing of the mouth; and
at least one spring member configured to bias the lever towards a closed position of the mouth; and
wherein, when the portion of the handle is received within the mouth, a ratio of torque resistance for rotation of the handle in a first direction relative to torque resistance for rotation of the handle in a second opposing direction is within a range of 0.8 to 1.2.

2. The delivery apparatus system of claim 1, wherein the first jaw member is a first lever rotatably attached to the base and the second jaw member is a stationary member which is attached to and non-moveable relative to the base.

3. The delivery apparatus system of claim 1, wherein the first curved interior wall has a decreased surface area relative to the second curved interior wall.

4. The delivery apparatus system of claim 1, wherein the first curved interior wall comprises a first upper lip defining a first side of an opening of the mouth and the second curved interior wall comprises a second upper lip defining a second side of the opening of the mouth, and wherein the first upper lip is recessed relative to the second upper lip.

5. The delivery apparatus system of claim 4, wherein the first curved interior wall further comprises a first lower lip opposing the first upper lip, and wherein the first curved interior wall has a surface gradient comprising a higher friction surface in a region of the first lower lip and a lower friction surface in a region the first upper lip.

6. The delivery apparatus system of claim 1, wherein one or the first curved interior wall or the second curved interior wall has a higher friction coefficient relative to the other of the first curved interior wall or the second curved interior wall.

7. The delivery apparatus system of claim 1, wherein the stabilizer clamp further comprises a lock configured to retain a position the lever such that the mouth is locked in the closed position.

8. The delivery apparatus system of claim 7, wherein the lever further comprises an extension configured to be depressed to rotate the lever to an open position of the mouth, and wherein the lock comprises an actuator on the extension for releasing the lock.

9. The delivery apparatus system of claim 1, wherein the first jaw member is a first lever and the second jaw member is a second lever, each of the first lever and the second lever rotatably attached to the base portion, and wherein the at least one spring comprises a first spring configured to bias the first lever towards the closed position of the mouth and a second spring member configured to bias the second lever towards the closed position of the mouth.

10. The delivery apparatus system of claim 9, wherein the first lever and the second lever are coupled to each other such that application of a rotational force towards the open position of the mouth on one of the first lever or the second lever to results in opening of the other of the first lever or the second lever.

11. The delivery apparatus system of claim 1, wherein, when the portion of the handle is received within the mouth, a minimum torque required for rotation of the handle in one or more of the first direction or the second opposing direction is in a range of 15 N-cm to 30 N-cm.

12. The delivery apparatus system of claim 1, wherein the ratio of torque resistance for rotation of the handle in a first direction relative to torque resistance for rotation of the handle in a second opposing direction is within a range of 0.9 to 1.1.

13. The delivery apparatus system of claim 12, wherein the ratio of torque resistance for rotation of the handle in a first direction relative to torque resistance for rotation of the handle in a second opposing direction is approximately 1.0.

14. A stabilizer clamp configured for use with an implant delivery apparatus, the stabilizer clamp comprising:
a base portion; and
a jaw portion extending from the base portion, the jaw portion comprising:
a first jaw member comprising a first curved interior wall; and
a second jaw member comprising a second curved interior wall, wherein the first curved interior wall and the second curved interior wall form a mouth configured to receive a portion of a handle of the implant delivery apparatus, and wherein at least one of the first jaw member or the second jaw member is a lever rotatable around a fulcrum for opening and closing of the mouth; and
at least one spring member configured to bias the lever towards a closed position of the mouth; and
wherein the stabilizer clamp is configured such that, when the portion of the handle is received within the mouth, a ratio of torque resistance for rotation of the handle in a first direction relative to torque resistance for rotation of the handle in a second opposing direction is within a range of 0.8 to 1.2.

15. The stabilizer clamp of claim 14, wherein the first jaw member is a first lever rotatably attached to the base and the second jaw member is a stationary member which is attached to and non-moveable relative to the base.

16. The stabilizer clamp of claim 15, wherein the stabilizer clamp further comprises a lock configured to retain a position the first lever such that the mouth is locked in the closed position.

17. The stabilizer clamp of claim 14, wherein the first curved interior wall comprises a first upper lip defining a first side of an opening of the mouth and the second curved interior wall comprises a second upper lip defining a second side of the opening of the mouth.

18. The stabilizer clamp of claim 17, wherein the first upper lip is recessed relative to the second upper lip, and the first curved interior wall has a decreased surface area relative to the second curved interior wall.

19. The stabilizer clamp of claim 17, wherein the first curved interior wall further comprises a first lower lip opposing the first upper lip, and wherein the first curved interior wall has a textured surface gradient comprising a high friction surface in a region of the first lower lip and a low friction surface in a region the first upper lip.

20. A method of operating a delivery apparatus system, the method comprising:
inserting a portion of a first handle of a first delivery apparatus into a mouth of a first stabilizer clamp, the first stabilizer clamp comprising a base portion attached to a stabilizer rail or table and a first jaw member and a second jaw member extending from the base portion, the mouth defined by a first curved interior wall of the first jaw member and a second curved interior wall of a second jaw member, wherein at least one of the first jaw member or the second jaw member is a lever rotatable around a fulcrum for opening and closing of the mouth, and wherein the first stabilizer clamp further comprises at least one spring member configured to bias the lever towards a closed position of the mouth;
rotating the first handle in a first direction to transmit torque to a first shaft coupled to the first handle; and
rotating the first handle in a second opposing direction to transit torque to the first shaft coupled to the first handle;
wherein the first stabilizer clamp is configured such that, when the portion of the first handle is received within the mouth thereof, a ratio of torque resistance for rotation of the first handle in the first direction relative to torque resistance for rotation of the first handle in the second opposing direction is within a range of 0.8 to 1.2.
